(19) United States
(12) Patent Application Publication
(10) Pub. No.: US 2004/0248109 A1
(43) Pub. Date: Dec. 9, 2004

(54) Methods for selecting protein binding moieties

(76) Inventor: Lawrence Greenfield, San Mateo, CA (US)

Correspondence Address: FINNEGAN, HENDERSON, FARABOW, GARRETT & DUNNER, LLP, 1300 I STREET, NW, WASHINGTON, DC, 20005, US

(21) Appl. No.: 10/457,943

(22) Filed: Jun. 9, 2003

(51) Int. Cl. C12Q 1/68; C12N 15/85

(52) U.S. Cl. 435/6; 435/455

(57) ABSTRACT

Methods, compositions, and apparatuses for the identification of binding moieties that bind to targets are provided. Vectors encoding potential binding moieties are also provided. In certain embodiments, methods are provided for the presentation of potential binding moieties by cells, the selection of binding moieties that bind targets, and the amplification of the nucleic acids encoding the binding moieties.

### FIELD OF THE INVENTION

Methods, compositions, and apparatuses for the identification of binding moieties that bind to targets are provided. Vectors encoding potential binding moieties are also provided. In certain embodiments, methods are provided for the presentation of potential binding moieties by cells, the selection of binding moieties that bind targets, and the amplification of the nucleic acids encoding the binding moieties.

### BACKGROUND

The detection and identification of polypeptides that bind various targets is known and useful. Exemplary areas in which such procedures may be used include therapeutics, drug validation, and basic research in the areas of cell signaling, receptor-ligand interaction, and drug discovery. Methods of identification of potential binding polypeptides include animal immunization, hybridoma technology, phage display and cell surface display. Those methods typically require successive rounds of amplification and screening which typically involve long periods of incubation for growth.

### SUMMARY OF THE INVENTION

In certain embodiments, methods of identifying potential binding moieties that bind to a target are provided. In certain embodiments, these methods comprise: incubating first host cells comprising vectors comprising a library of nucleic acids encoding potential binding moieties to express potential binding moieties such that the binding moieties are presented on the surface of the first host cells; exposing the first host cells with potential binding moieties on their surface to targets; and selecting at least one first host cell with a potential binding moiety on its surface bound to a target. In certain embodiments, these methods further comprise allowing the nucleic acid encoding the potential binding moiety to amplify in a manner that does not require cell division, said amplifying comprising: exposing second host cells, that do not comprise nucleic acids encoding the potential binding moieties, to a selected at least one first host cell; and transmitting the nucleic acid encoding the potential binding moiety to at least one second host cell. In certain embodiments, these methods further comprise identifying the potential binding moiety that binds the target.

In certain embodiments, after the nucleic acid encoding the potential binding moiety is transmitted to the second host cells, the second host cells are subjected to a subsequent round of a selection comprising: incubating the second host cells to express potential binding moieties such that the potential binding moieties are presented on the surface of the second host cells; exposing the second host cells with potential binding moieties on their surface to targets; and selecting at least one second host cell with a potential binding moiety on its surface bound to a target.

In certain embodiments, methods of identifying potential binding moieties that bind to a target are provided. In certain embodiments, these methods comprise: incubating first host cells comprising vectors comprising a library of nucleic acids encoding potential binding moieties to express the potential binding moieties such that the potential binding moieties are presented in an accessible compartment of the first host cells; exposing the first host cells with potential binding moieties in their accessible compartments to targets; and selecting at least one first host cell with a potential binding moiety bound to a target. In certain embodiments, the methods further comprise allowing the nucleic acid encoding the potential binding moiety to amplify in a manner that does not require cell division, said amplifying comprising: exposing second host cells, that do not comprise nucleic acids encoding the potential binding moieties, to a selected at least one first host cell; and transmitting the nucleic acid encoding the potential binding moiety to the second host cells; and identifying the potential binding moiety that binds the target.

In certain embodiments, after the nucleic acid encoding the potential binding moiety is transmitted to the second host cells, the second host cells are subjected to a subsequent round of a selection comprising: incubating the second host cells to express potential binding moieties such that the potential binding moieties are presented in the accessible compartment of the second host cells; exposing the second host cells with potential binding moieties in their accessible compartments to targets; and selecting at least one second host cell with a potential binding moiety in its accessible compartment bound to a target.

In certain embodiments, methods of identifying potential binding moieties that bind to a target are provided. In certain embodiments, the methods comprise: incubating first host cells comprising vectors comprising a library of nucleic acids encoding potential binding moieties to express the potential binding moieties such that the binding moieties are presented on the surface of the first host cells; exposing the first host cells with potential binding moieties on their surface to targets; and selecting at least one first host cell with a potential binding moiety on its surface bound to a target. In certain embodiments, the methods further comprise allowing the nucleic acid encoding the potential binding moiety to amplify, said amplifying comprising exposing a culture of second host cells, that do not comprise nucleic acids encoding the potential binding moieties, to a selected at least one first host cell; and transmitting the nucleic acid encoding the potential binding moiety to second host cells. In certain embodiments, the methods further comprise identifying the potential binding moiety that binds the target.

In certain embodiments, after the nucleic acid encoding the potential binding moiety is transmitted to the second host cells, the second host cells are subjected to a subsequent round of a selection comprising: incubating the second host cells to express the potential binding moieties such that the binding moieties are presented on the surface of the second host cells; exposing the second host cells with potential binding moieties on their surface to targets; and selecting at least one second host cell with a potential binding moiety on its surface bound to a target.

In certain embodiments, methods of identifying potential binding moieties that bind to a target are provided. In certain embodiments, the methods comprise: incubating first host cells comprising vectors comprising a library of nucleic acids encoding potential binding moieties to express the potential binding moieties such that the potential binding moieties are presented in accessible compartments of the first host cells; exposing the first host cells with potential binding moieties in their accessible compartments to targets; and selecting at least one first host cell with a potential binding moiety bound to a target. In certain embodiments, the methods further comprise allowing the nucleic acid encoding the potential binding moiety to amplify, said amplifying comprising: exposing a culture of second host cells, that do not comprise nucleic acids encoding the potential binding moieties, to a selected at least one first host cell; and transmitting the nucleic acid encoding the potential binding moiety to the second host cells. In certain embodiments, the methods further comprise identifying the potential binding moiety that binds the target.

In certain embodiments, after the nucleic acid encoding the potential binding moiety is transmitted to the second host cells are, the second host cells are subjected to a subsequent round of a selection comprising: incubating the second host cells to express the potential binding moieties such that the potential binding moieties are presented in the accessible compartment of the second host cells; exposing the second host cells with potential binding moieties in their accessible compartments to targets; and selecting at least one second host cell with a potential binding moiety in its accessible compartment bound to a target.

In certain embodiments, vectors encoding a polypeptide are provided. In certain embodiments, the polypeptide comprises: at least one of a secretory leader and a carrier protein that permits secretion of at least a portion of the polypeptide into an accessible compartment or an extracellular space when expressed in a host cell; a potential binding moiety to be tested for binding to a target; and an anchor portion that attaches at least a portion of the polypeptide to either an inner membrane or an outer membrane of a host cell, wherein when the vector is included in a host cell, the host cell is capable of transmitting the vector to another host cell; and wherein the vector comprises at least one element for transmission of nucleic acid.

In certain embodiments, a transmitting agent comprising the vector is provided. In certain embodiments, a phage comprising the vector is provided. In certain embodiments, a library comprising multiple vectors is provided, wherein each vector comprises different nucleic acid sequences encoding different potential binding moieties.

In certain embodiments, vectors encoding a polypeptide are provided. In certain embodiments, the polypeptide comprises: at least one of a secretory leader and a carrier protein that permits secretion of the polypeptide into an accessible compartment when expressed in a host cell; a potential binding moiety to be tested for binding to a target; and a signal portion that directs transportation of the polypeptide to the accessible compartment of a host cell, wherein when the vector is included in a host cell, the host cell is capable of transmitting the vector to another host cell; and wherein the vector comprises at least one element for transmission of nucleic acid.

In certain embodiments, a transmitting agent comprising the vector is provided. In certain embodiments, a phage comprising the vector is provided. In certain embodiments, a library comprising multiple vectors is provided, wherein each vector comprises different nucleic acid sequences encoding different potential binding moieties.

In certain embodiments, vectors encoding a polypeptide are provided. In certain embodiments, the polypeptide comprises: a secretory leader that permits secretion of at least a portion of the polypeptide into an accessible compartment or an extracellular space when expressed in a host cell; a leader peptidase cleavage site that allows the secretory leader to be cleaved from the polypeptide in the presence of a peptidase; a potential binding moiety to be tested for binding to a target; a scaffold peptide that provides a secondary structure to the potential binding moiety; an anchor portion that attaches a portion of the polypeptide to either an inner membrane or an outer membrane of a host cell; and a linker peptide disposed between the potential binding moiety and the anchor portion. In certain embodiments, the vector further comprises: a regulatory region selected from at least one of an operator and an initiator; a promoter region that allows binding of an RNA polymerase; a transcription initiation site; a ribosomal binding site comprising a Shine-Delgarno sequence; a start codon; a termination codon; and a transcriptional terminator; wherein when the vector is included in a host cell, the host cell is capable of transmitting the vector to another host cell; and wherein the vector comprises at least one element for transmission of nucleic acid.

In certain embodiments, an apparatus is provided, comprising: a component that exposes first bacterial cells with potential binding moieties to targets; a component that separates at least one first host cell with a potential binding moiety bound to a target from the first host cells that are not bound to targets; and a component that exposes a culture of host cells lacking nucleic acids encoding potential binding moieties to the selected at least one first host cell.

### BRIEF DESCRIPTION OF THE FIGURES

FIGS. 1A-G illustrate steps of certain exemplary methods according to certain embodiments. Each of the letters Q to Z in FIGS. 1C to 1G represent a host cell with a different potential binding moiety presented. FIG. 1H illustrates the cycle of exemplary methods according to certain embodiments.

FIGS. 2A-C illustrate certain exemplary modes of presentation of potential binding moieties on inner and outer membranes according to certain embodiments. FIGS. 2D and 2E illustrate certain exemplary modes of presentation of potential binding moieties in accessible compartments according to certain embodiments.

FIG. 3 illustrates an exemplary vector according to certain embodiments.

FIG. 4 illustrates certain exemplary methods of selecting binding moieties of different affinities according to certain embodiments.

FIG. 5 illustrates an exemplary cycle of selection in certain phage display techniques.

FIG. 6 illustrates an exemplary transmitting agent, a bacteriophage M13, according to certain embodiments.

FIG. 7 illustrates an exemplary apparatus according to certain embodiments.

FIG. 8 illustrates an exemplary apparatus according to certain embodiments.

### DETAILED DESCRIPTION OF CERTAIN EMBODIMENTS OF THE INVENTION

It is to be understood that both the foregoing general description and the following detailed description are exemplary and explanatory only and are not restrictive of the invention, as claimed. In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

Standard techniques may be used for recombinant DNA, oligonucleotide synthesis, and tissue culture and transformation (e.g., chemical, electroporation, lipofection, etc.). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)), which is incorporated herein by reference for any purpose. Unless specific definitions are provided, the nomenclatures utilized in connection with, and the laboratory procedures and techniques of, analytical chemistry, synthetic organic chemistry, and medicinal and pharmaceutical chemistry described herein are those well known and commonly used in the art. Standard techniques may be used for chemical syntheses, chemical analyses, pharmaceutical preparation, formulation, and delivery.

Definitions and Terms

The term “nucleotide base”, as used herein, refers to a substituted or unsubstituted aromatic ring or rings. In certain embodiments, the aromatic ring or rings contain at least one nitrogen atom. In certain embodiments, the nucleotide base is capable of forming Watson-Crick and/or Hoogsteen hydrogen bonds with an appropriately complementary nucleotide base. Exemplary nucleotide bases and analogs thereof include, but are not limited to, naturally occurring nucleotide bases adenine, guanine, cytosine, uracil, thymine, and analogs of the naturally occurring nucleotide bases, e.g., 7-deazaadenine, 7-deazaguanine, 7-deaza-8-azaguanine, 7-deaza-8-azaadenine, N6-Δ2-isopentenyladen ine (6iA), N6-Δ2-isopentenyl-2-methylthioadenine (2 ms6iA), N2-dimethylguanine (dmG), 7-methylguanine (7mG), inosine, nebularine, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine, 7-deazaguanine, 2-thiopyrimidine, 6-thioguanine, 4-thiothymine, 4-thiouracil, O⁶-methylguanine, N⁶-methyladenine, O⁴-methylthymine, 5,6-dihydrothymine, 5,6-dihydrouracil, pyrazolo[3,4-D]pyrimidines (see, e.g., U.S. Pat. Nos. 6,143,877 and 6,127,121 and PCT published application WO 01/38584), ethenoadenine, indoles such as nitroindole and 4-methylindole, and pyrroles such as nitropyrrole. Certain exemplary nucleotide bases can be found, e.g., in Fasman, 1989, Practical Handbook of Biochemistry and Molecular Biology, pp. 385-394, CRC Press, Boca Raton, Fla., and the references cited therein.

The term “nucleotide”, as used herein, refers to a compound comprising a nucleotide base linked to the C-1′ carbon of a sugar, such as ribose, arabinose, xylose, and pyranose, and sugar analogs thereof. The term nucleotide also encompasses nucleotide analogs. The sugar may be substituted or unsubstituted. Substituted ribose sugars include, but are not limited to, those riboses in which one or more of the carbon atoms, for example the 2′-carbon atom, is substituted with one or more of the same or different Cl, F, —R, —OR, —NR₂ or halogen groups, where each R is independently H, C₁-C₆ alkyl or C₅-C₁₄ aryl. Exemplary riboses include, but are not limited to, 2′-(C1-C6)alkoxyribose, 2′-(C5-C14)aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C1-C6)alkylribose, 2′-deoxy-3′-(C1-C6)alkoxyribose and 2′-deoxy-3′-(C5-C14)aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-α-anomeric nucleotides, 1′-α-anomeric nucleotides, 2′-4′- and 3′-4′-linked and other “locked” or “LNA”, bicyclic sugar modifications (see, e.g., PCT published application nos. WO 98/22489, WO 98/39352; and WO 99/14226). Exemplary LNA sugar analogs within a polynucleotide include, but are not limited to, the structures:

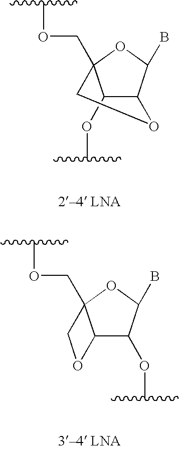

where B is any nucleotide base.

Modifications at the 2′- or 3′-position of ribose include, but are not limited to, hydrogen, hydroxy, methoxy, ethoxy, allyloxy, isopropoxy, butoxy, isobutoxy, methoxyethyl, alkoxy, phenoxy, azido, amino, alkylamino, fluoro, chloro and bromo. Nucleotides include, but are not limited to, the natural D optical isomer, as well as the L optical isomer forms (see, e.g., Garbesi (1993) Nucl. Acids Res. 21:4159-65; Fujimori (1990) J. Amer. Chem. Soc. 112:7435; Urata, (1993) Nucleic Acids Symposium Ser. No. 29:69-70). When the nucleotide base is purine, e.g. A or G, the ribose sugar is attached to the N⁹-position of the nucleotide base. When the nucleotide base is pyrimidine, e.g. C, T or U, the pentose sugar is attached to the N¹-position of the nucleotide base, except for pseudouridines, in which the pentose sugar is attached to the C5 position of the uracil nucleotide base (see, e.g., Kornberg and Baker, (1992) DNA Replication, 2^{nd} Ed., Freeman, San Francisco, Calif.).

One or more of the pentose carbons of a nucleotide may be substituted with a phosphate ester having the formula:

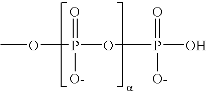

where α is an integer from 0 to 4. In certain embodiments, α is 2 and the phosphate ester is attached to the 3′- or 5′-carbon of the pentose. In certain embodiments, the nucleotides are those in which the nucleotide base is a purine, a 7-deazapurine, a pyrimidine, or an analog thereof. “Nucleotide 5″-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group may include sulfur substitutions for the various oxygens, e.g. α-thio-nucleotide 5′-triphosphates. For a review of nucleotide chemistry, see: Shabarova, Z. and Bogdanov, A. Advanced Organic Chemistry of Nucleic Acids, VCH, New York, 1994.

The term “nucleotide analog”, as used herein, refers to embodiments in which the pentose sugar and/or the nucleotide base and/or one or more of the phosphate esters of a nucleotide may be replaced with its respective analog. In certain embodiments, exemplary pentose sugar analogs are those described above. In certain embodiments, the nucleotide analogs have a nucleotide base analog as described above. In certain embodiments, exemplary phosphate ester analogs include, but are not limited to, alkylphosphonates, methylphosphonates, phosphoramidates, phosphotriesters, phosphorothioates, phosphorodithioates, phosphoroselenoates, phosphorodiselenoates, phosphoroanilothioates, phosphoroanilidates, phosphoroamidates, boronophosphates, etc., and may include associated counterions.

Also included within the definition of “nucleotide analog” are nucleotide analog monomers which can be polymerized into polynucleotide analogs in which the DNA/RNA phosphate ester and/or sugar phosphate ester backbone is replaced with a different type of internucleotide linkage. Exemplary polynucleotide analogs include, but are not limited to, peptide nucleic acids, in which the sugar phosphate backbone of the polynucleotide is replaced by a peptide backbone.

As used herein, the terms “polynucleotide”, “oligonucleotide”, and “nucleic acid” are used interchangeably and mean single-stranded and double-stranded polymers of nucleotide monomers, including 2′-deoxyribonucleotides (DNA) and ribonucleotides (RNA) linked by internucleotide phosphodiester bond linkages, or internucleotide analogs, and associated counter ions, e.g., H⁺, NH₄+, trialkylammonium, Mg²⁺, Na⁺ and the like. A nucleic acid may be composed entirely of deoxyribonucleotides, entirely of ribonucleotides, or chimeric mixtures thereof. The nucleotide monomer units may comprise any of the nucleotides described herein, including, but not limited to, naturally occuring nucleotides and nucleotide analogs nucleic acids typically range in size from a few monomeric units, e.g. 5-40 when they are sometimes referred to in the art as oligonucleotides, to several thousands of monomeric nucleotide units. Unless denoted otherwise, whenever a nucleic acid sequence is represented, it will be understood that the nucleotides are in 5′ to 3′ order from left to right and that “A” denotes deoxyadenosine or an analog thereof, “C” denotes deoxycytidine or an analog thereof, “G” denotes deoxyguanosine or an analog thereof, and “T” denotes thymidine or an analog thereof, unless otherwise noted.

Nucleic acids include, but are not limited to, genomic DNA, cDNA, hnRNA, mRNA, rRNA, tRNA, fragmented nucleic acid, synthetic nucleic acid, nucleic acid obtained from subcellular organelles such as mitochondria or chloroplasts, and nucleic acid obtained from microorganisms or DNA or RNA viruses that may be present on or in a biological sample.

Nucleic acids may be composed of a single type of sugar moiety, e.g., as in the case of RNA and DNA, or mixtures of different sugar moieties, e.g., as in the case of RNA/DNA chimeras. In certain embodiments, nucleic acids are ribopolynucleotides and 2′-deoxyribopolynucleotides according to the structural formulae below:

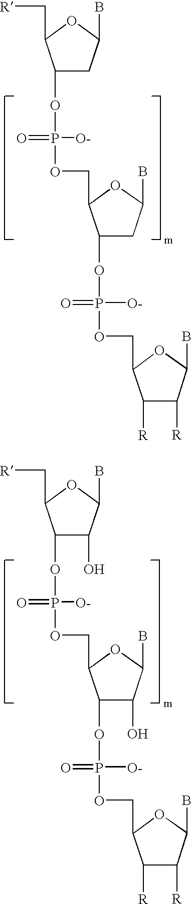

wherein each B is independently the base moiety of a nucleotide, e.g., a purine, a 7-deazapurine, a pyrimidine, or an analog nucleotide; each m defines the length of the respective nucleic acid and can range from zero to thousands, tens of thousands, or even more; each R is independently selected from the group comprising hydrogen, halogen, —R″, —OR″, and —NR″R″, where each R″ is independently (C1-C6) alkyl or (C5-C14) aryl, or two adjacent Rs are taken together to form a bond such that the ribose sugar is 2′,3′-didehydroribose; and each R′ is independently hydroxyl or

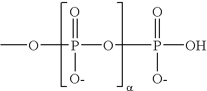

where α is zero, one or two.

In certain embodiments of the ribopolynucleotides and 2′-deoxyribopolynucleotides illustrated above, the nucleotide bases B are covalently attached to the C1′ carbon of the sugar moiety as previously described.

The term “oligonucleotide” referred to herein includes naturally occurring, and modified nucleotides linked together by naturally occurring, and/or non-naturally occurring oligonucleotide linkages. Oligonucleotides are a polynucleotide subset generally comprising a length of 200 bases or fewer. In certain embodiments, oligonucleotides are 10 to 60 bases in length. In certain embodiments, oligonucleotides are 12, 13, 14, 15, 16, 17, 18, 19, or 20 to 40 bases in length. Oligonucleotides may be single stranded or double stranded, e.g. for use in the construction of a gene mutant. Oligonucleotides of the invention may be sense or antisense oligonucleotides.

The term “naturally occurring nucleotides” includes deoxyribonucleotides and ribonucleotides. The term “modified nucleotides” includes nucleotides with modified or substituted sugar groups and the like. The term “oligonucleotide linkages” includes oligonucleotides linkages such as phosphorothioate, phosphorodithioate, phosphoroselenoate, phosphorodiselenoate, phosphoroanilothioate, phoshoraniladate, phosphoroamidate, and the like. See, e.g., LaPlanche et al. Nucl. Acids Res. 14:9081 (1986); Stec et al. J. Am. Chem. Soc. 106:6077 (1984); Stein et al. Nucl. Acids Res. 16:3209 (1988); Zon et al. Anti-Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990), the disclosures of which are hereby incorporated by reference for any purpose. An oligonucleotide can include a label for detection.

The terms “nucleic acid”, “polynucleotide”, and “oligonucleotide” may also include nucleic acid analogs, polynucleotide analogs, and oligonucleotide analogs. The terms “nucleic acid analog”, “polynucleotide analog” and “oligonucleotide analog” are used interchangeably and, as used herein, refer to a nucleic acid that contains at least one nucleotide analog and/or at least one phosphate ester analog and/or at least one pentose sugar analog. Also included within the definition of nucleic acid analogs are nucleic acids in which the phosphate ester and/or sugar phosphate ester linkages are replaced with other types of linkages, such as N-(2-aminoethyl)-glycine amides and other amides (see, e.g., Nielsen et al., 1991, Science 254: 1497-1500; WO 92/20702; U.S. Pat. No. 5,719,262; U.S. Pat. No. 5,698,685); morpholinos (see, e.g., U.S. Pat. No. 5,698,685; U.S. Pat. No. 5,378,841; U.S. Pat. No. 5,185,144); carbamates (see, e.g., Stirchak & Summerton, 1987, J. Org. Chem. 52: 4202); methylene(methylimino) (see, e.g., Vasseur et al., 1992, J. Am. Chem. Soc. 114: 4006); 3′-thioformacetals (see, e.g., Jones et al., 1993, J. Org. Chem. 58: 2983); sulfamates (see, e.g., U.S. Pat. No. 5,470,967); 2-aminoethylglycine, commonly referred to as PNA (see, e.g., Buchardt, WO 92/20702; Nielsen (1991) Science 254:1497-1500); and others (see, e.g., U.S. Pat. No. 5,817,781; Frier & Altman, 1997, Nucl. Acids Res. 25:4429 and the references cited therein). Phosphate ester analogs include, but are not limited to, (i) C₁-C₄ alkylphosphonate, e.g. methylphosphonate; (ii) phosphoramidate; (iii) C₁-C₆ alkyl-phosphotriester; (iv) phosphorothioate; and (v) phosphorodithioate.

The terms “annealing” and “hybridization” are used interchangeably and mean the base-pairing interaction of one nucleic acid with another nucleic acid that results in formation of a duplex, triplex, or other higher-ordered structure. In certain embodiments, the primary interaction is base specific, e.g., A/T and G/C, by Watson/Crick and Hoogsteen-type hydrogen bonding. In certain embodiments, base-stacking and hydrophobic interactions may also contribute to duplex stability.

As used herein, the terms “peptide,” “polypeptide,” and “protein” are used interchangeably and mean polymers of amino acid monomers linked by polypeptide linkages between carboxyl (COOH) groups and amine (NH₄) groups. A peptide may consist entirely of naturally occurring amino acid monomers, non-naturally occurring amino acids, or chimeric mixtures thereof. Unless denoted otherwise, whenever an amino acid sequence is represented, it will be understood that the amino acids are in N-terminal to C-terminal order from left to right. The term “polypeptide” may refer to small peptides, larger polypeptides, proteins containing single polypeptide chains, proteins containing multiple polypeptide chains, and multi-subunit proteins.

Alterations to the amino acids may include, but are not limited to, glycosylation, methylation, phosphorylation, biotinylation, and any covalent and noncovalent additions to a protein that do not result in a change in amino acid sequence. “Amino acid” as used herein refers to any amino acid, natural or nonnatural, that may be incorporated, either enzymatically or synthetically, into a polypeptide or protein.

The terms “target” and “target molecule,” as used herein, refer to any molecule of interest for which a binding moiety is sought. Exemplary targets include, but are not limited to, secreted peptide growth factors, pharmaceutical agents, cell signaling molecules, blood proteins, portions of cell surface receptor molecules, portions of nuclear receptors, steroid molecules, viral proteins, antibodies, portions of antibodies, carbohydrates, enzymes, active sites of enzymes, binding sites of enzymes, portions of enzymes, small molecules, drugs, small molecule drugs, cells, bacterial cells, proteins, epitopes of proteins, surfaces of proteins involved in protein-protein interactions, cell surface eptiopes, diagnostic proteins, diagnostic markers, peptides encoded by open reading frames, plant proteins, peptides involved in protein-protein interactions, and foods.

The term “label” as used herein refers to any tag, marker, or identifiable moiety. The skilled artisan will appreciate that many labels may be used in the present invention. For example, labels include, but are not limited to, fluorophores, radioisotopes, chromogens, enzymatic labels, antigens, heavy metals, dyes, magnetic probes, magnetic particles, paramagnetic particles, electrophoretic molecules and particles, dielectrophoretic particles, phosphorescence groups, members of affinity binding sets, and electrochemical detection moieties, chemiluminescent, biotinyl groups, predetermined polypeptide epitopes recognized by another moeity (e.g., leucine zipper pair sequences, binding sites for secondary antibodies, metal binding domains, epitope tags), mobility modifiers, and particles that confer a dielectrophoretic change. Exemplary fluorophores that are used as labels include, but are not limited to, lanthanide phosphors, FITC, rhodamine, cyanine 3 (Cy 3), cyanine 5 (Cy 5), fluorescein, Vic™, Liz™, Tamra™, 5-Fam™, 6-Fam™, and Texas Red (Molecular Probes). (Vic™, Liz™, Tamra™, 5-Fam™, and 6-Fam™ are all available from Applied Biosystems, Foster City, Calif.) Exemplary radioisotopes include, but are not limited to, ³H, ¹⁴C, ¹⁵N, ³⁵S, ⁹⁰Y, 99 Tc, ¹¹¹In, ¹²⁵I, ¹³¹I ³²P, and ³³P. Exemplary enzymatic labels include, but are not limited to, horseradish peroxidase, β-galactosidase, luciferase, alkaline phosphatase. Exemplary affinity binding sets include, but not limited to, biotin/avidin, antibody/antigen, biotin/streptavidin, hexahistidine/nickel-NTA, pentahistidine/nickel-NTA, hexahistidine/anti-hexahistidine, e-tag/anti-e-tag antibody, calmodulin-binding peptide/calmodulin, fluorescein/antifluorescein antibody, digoxygenin/anti-digoxygenin antibody, ligand/receptor, enzyme/substrate, and the like, in which one member interacts with other members of the set in order to effect a detectable signal. One exemplary affinity binding set includes a biotin label attached to a target and an avidin moiety conjugated with a fluorescent label. The skilled artisan will appreciate that, in certain embodiments, one or more of the targets, potential binding moieties, or other moieties bound to either targets or potential binding moieties disclosed herein may further comprise one or more labels. Various methods of labeling polypeptides and glycoproteins are known in the art and may be used.

As used herein, the term “separating label” refers to a label employed to separate a molecule attached to a separating label from molecules not attached to a separating label. Exemplary separating labels include, but are not limited to, members of affinity binding sets, mobility modifiers, magnetic particles, predetermined polypeptide epitopes recognized by another moiety (e.g., leucine zipper pair sequences, binding sites for secondary antibodies, metal binding domains, epitope tags), mobility modifiers, latex particles, paramagnetic particles, particles with specific dielectrophoretic properties, charged particles, and particles that confer a dielectrophoretic change. In certain embodiments, separating labels may interact with other moieties to facilitate separation, e.g., a biotin label attached to a target molecule may bind to a streptavidin moiety attached to a particle to effect separation of the target molecule from other molecules.

“Detectably different,” as used herein, means that detectable signals from different labels are distinguishable from one another by at least one detection method. As used herein, detection methods include, but are not limited to, magnetic and paramagnetic field resolution, diamagnetic resolution, electrophorectic resolution, dielectrophoretic resolution, fluorescent activated cell sorting (FACS), fluorescence detection, and colorimetric detection.

As used herein, a particle “coated” with a substance refers to a particle with a substance attached to the surface as well as a particle with a substance embedded in the particle. In certain embodiments, a particle coated with a substance is a particle with a substance on part of the surface. In certain embodiments, a particle coated with a substance is a particle with a substance on the entire surface.

The term “binding moiety,” as used herein, refers to any moiety that demonstrates a detectable ability to bind to a target. A detectable ability to bind a target includes, but is not limited to, the ability to detect the binding of a binding moiety indirectly through another moiety other than the binding moiety. Several types of binding moieties have been characterized, including, but not limited to, portions of receptors, antibodies, enzymes, polypeptides, carbohydrates, polysaccharides, other small molecules, and any component that can be presented in an accessible compartment or on a surface of a host cell. In certain embodiments, not all components of a binding moiety may be necessary for the binding moiety to bind a target. In certain embodiments, substitutions of certain components within a binding moiety may also permit a binding moiety to retain the capacity to bind a target.

Further, a “specific binding moiety” refers to a binding moiety that demonstrates an ability to specifically bind one or more targets. While a “specific binding moiety” may bind to molecules other than the one or more targets, the affinity of the specific binding moiety for molecules other than the one or more targets is significantly lower than the affinity of the specific binding moiety for the one or more targets. In certain embodiments, the affinity of a specific binding moiety for one or more targets is at least 5% greater than the affinity of the specific binding moiety for molecules other than the one or more targets. In certain embodiments, the affinity of a specific binding moiety for one or more targets is at least 10% greater than the affinity of the specific binding moiety for molecules other than the one or more targets. In certain embodiments, the affinity of a specific binding moiety for one or more targets is at least 15% greater than the affinity of the specific binding moiety for molecules other than the one or more targets. In certain embodiments, the affinity of a specific binding moiety for one or more targets is at least 20% greater than the affinity of the specific binding moiety for molecules other than the one or more targets. Conversely, a “non-specific binding moiety” is a moiety that possesses an affinity for both target as well as non-target molecules, wherein the affinity of the non-specific binding moiety for one or more targets is not significantly different from the affinity of the non-specific binding moiety for molecules other than the one or more targets.

The term “antibody” refers to an intact antibody or fragment, or derivatives of either one or both chains of an antibody thereof that specifically binds to an antigen. In certain embodiments, antibody fragments are produced by recombinant DNA techniques. In certain embodiments, antibody fragments are produced by enzymatic or chemical cleavage of intact antibodies. In certain embodiments, antibody fragments are produced synthetically. In certain embodiments, antibody fragments are produced chemically. Antibody fragments include, but are not limited to, Fab, Fab′, F(ab′)₂, Fv, single-chain antibodies, and single domain antibodies.

As used herein, an “affinity binding set” is a set of molecules that specifically bind to one another. Affinity binding sets include, but are not limited to, biotin and avidin, biotin and streptavidin, receptor and ligand, antibody and ligand, antibody and antigen, calmodulin and calmodulin-binding protein, hexa-histidine and nickel nitriloacetic acid (Ni-NTA), penta-histidine and nickel nitriloacetic acid (Ni-NTA), and a polynucleotide sequence and its complement. In certain embodiments, affinity binding sets comprise an antibody and an epitope tag. In certain embodiments, an epitope tag is a peptide that is recognized by a known antibody. In certain embodiments, affinity binding sets that are bound may be unbound. For example, in certain embodiments, polynucleotide sequences that are hybridized may be denatured, and biotin bound to streptavidin may be heated and become unbound. In certain embodiments, antibodies may be released from their target by lowering the pH or increasing the magnesium concentration. In certain embodiments, streptavidin bound to 2-iminobiotin may be released by lowering the pH.

As used herein, the term “library” refers to any collection of different nucleic acids. In certain embodiments, members of a library may be characterized by their biological origin, for example, a library may be generated representing the mRNA expressed in a nonimmunized spleen. In certain embodiments, a library may be a collection of nucleic acids encoding random peptides. Random peptides may also be modified to favor the inclusion of certain amino acid residues, or peptides of certain lengths. In certain embodiments, the term “library” may refer to a collection of different nucleic acid sequences included in vectors. In certain embodiments, different nucleic acid sequences are included in separate vectors. In certain embodiments, the separate vectors are identical except for the different nucleic acid sequence included in them. In certain embodiments, the term “library” may also refer to a collection of different nucleic acids not contained in vectors. In certain embodiments, the term “library” may refer to a collection of different nucleic acid sequences included in separate vectors. In certain embodiments, the separate vectors are identical except for the different nucleic acid sequences included in them.

As used herein, a “blocking molecule” refers to a molecule that is used to in a process to reduce the amount of nonspecific binding moieties selected. For example, in certain embodiments, if one is attempting to find a binding moiety that specifically binds a phosphorylated protein, an unphosphorylated protein may be used as a blocking molecule. In such an embodiment, the unphosphorylated protein binds nonspecific potential binding moieties that may compete for binding to the phosphorylated protein. Another example may be the inclusion of free streptavidin as a “blocking molecule” during the selection of a host cell to reduce the selection of non-specific binding moieties when streptavidin-coated particles are used during a separation step. Another example may be the inclusion of a normal cell as a “blocking molecule” during the selection of binding moieties against a diseased cell to identify specific binding moieties that bind to molecules uniquely present on the diseased cell. Use of a blocking molecule for selection of specific potential binding moieties over nonspecific binding moieties that interact with the blocking molecule may be referred to as “negative selection.”

As used herein, the term nucleic acid “amplification” refers to replicating at least one copy of a nucleic acid, and transmitting the nucleic acid to a cell that lacks the nucleic acid.

As used herein, nucleic acid amplification “in a manner that does not require cell division” refers to a method of nucleic acid amplification that is not the result of mitotic cell division. Nucleic acid amplification in a manner that does not require cell division does not exclude methods in which cell division occurs along with amplification that is not the result of cell division. Exemplary methods include, but are not limited to, phage amplification and assembly, phage assembly through use of a helper phage, transfection, conjugation, transformation, and transduction.

Nucleic acid amplification with “insubstantial cell division” refers to nucleic acid amplification in which the cells do not undergo cell division for a substantial amount of time. In certain embodiments, the cells do not undergo cell division for more than ten hours. In certain embodiments, the cells do not undergo cell division for more than six hours. In certain embodiments, the cells do not undergo cell division for more than four hours. In certain embodiments, the cells do not undergo cell division for more than three hours. In certain embodiments, the cells do not undergo cell division for more than two hours. In certain embodiments, the cells do not undergo cell division for more than one hour.

As used herein, “transmitting” nucleic acid from one cell to another refers to any method where nucleic acid in one cell ends up in another cell. Exemplary methods include both active methods of inducing transmission employing external manipulation, and methods where the nucleic acid is transmitted without any external manipulation. Such methods include, but are not limited to, phage assembly and infection; phage assembly through use of a helper phage coupled with infection; transduction; conjugation; transformation; and transfection.

As used herein, “at least one element for transmission of nucleic acid” refers to at least one nucleic acid that is employed in transmitting nucleic acid from one cell to another, or a nucleic acid that encodes at least one polypeptide employed in transmitting nucleic acid from one cell to another. Such elements include, but are not limited to, nucleic acids that encode phage coat proteins, nucleic acids that encode proteins necessary for phage assembly, regulatory elements that allow for rolling phage replication, and f+ pillus elements that allow for bacterial conjugation. Vectors that comprise “at least one element for transmission of nucleic acid” may include, but are not limited to, phage vectors and phagemids that assemble phages in the presence of a helper phage, F factors, sex factors, episomes, and plasmids. Exemplary phage vectors include, but are not limited to, vectors that encode at least a portion of M13, vectors that encode at least a portion of non-lytic forms of lambda phage, vectors that encode at least a portion of herpes virus, and vectors that encode at least a portion of cytomegalovirus.

As used herein, the term “on the surface” of a cell refers to the outer surface of a cell. In certain embodiments, when a potential binding moiety is presented “on the surface” of a cell, it remains associated with the cell. In certain embodiments, a polypeptide is attached to the surface of a cell because a portion of the polypeptide remains within the outer membrane of the cell. In certain embodiments, a portion of a polypeptide presented on the surface of the cell remains inside the outer membrane.

As used herein, the term “accessible compartment” of a host cell refers to a part of a cell, where a target may bind a potential binding moiety. In certain embodiments, the accessible compartment is the surface of the outer membrane of a cell. In certain embodiments, the potential binding moiety may be in a soluble form, yet still be associated with a host cell in an accessible compartment, provided the potential binding moiety remains inside the cell. Potential binding moieties in accessible compartments, for example, may be attached to the inner portion of the outer membrane of bacterial cells, the outer portion of the inner membrane of bacterial cells, the inner portion of the inner membrane of bacterial cells, or the potential binding moieties may be soluble within the intracellular space of a cell. Exemplary accessible compartments include, but are not limited to, the periplasmic space of bacterial cells, cytoplasmic space, endosomes, and vacuoles of eukaryotic cells.

As used herein, the term “type” with respect to cells refers to genetically distinct strains of cells. In certain embodiments, the genetic distinction between two cell types may be a single gene. In certain embodiments, the genetic distinction between two cell types may be the presence of certain extrachromosomal elements in one cell type, but not in another cell type. In certain embodiments, different types of cells may be able to perform the same functions, but may be used for slightly different purposes. For example, in certain embodiments, one cell type may perform better at producing a protein, while another type of cell may be better at producing phages when infected.

The terms “virus” and “phage” are used interchangeably in this application.

The term “employing a helper phage” refers to the use of a phage that encodes proteins that are employed in amplification. In certain embodiments, the proteins encoded by the helper phage are used in the assembly and production of phage and/or the replication of vectors. In certain embodiments, the absence of a helper phage prevents the replication of vectors or the assembly of phage.

The terms “viral assembly” and “phage assembly” refer to the assembly of proteins and nucleic acids into virus and phage particles that are capable of infecting cells with the nucleic acid. As used herein, the terms “viral assembly” and “phage assembly” are used interchangeably.

The term “transmitting agent” refers to an agent that assists in transmitting nucleic acids into a cell. Exemplary transmitting agents include, but are not limited to phage; vectors that encode proteins that are involved in conjugation, for example, but not limited to, sex factors, F factors, episomes, and other extrachromosomal elements; and chemical agents that facilitate the transfer of vectors into a cell. Exemplary chemicals that may act as transmitting agents include, but are not limited to, calcium chloride, rubidium chloride, manganese chloride, potassium chloride, dimethylsulfoxide, cobalt hexamine, calcium phosphate, DEAE dextran, liposomes, cationic lipids, poly-L-lysine, dendrimers, galanin-mastoparan chimeric sequences, nuclear localization sequences, and other peptide-based cellular transporters.

As used herein, the term “replication” refers to the replication of nucleic acid. In certain embodiments, replication refers to the replication of vectors. Replication of nucleic acid includes, but is not limited to, plasmid replication, episome replication, F′ replication, phage replication, and the replication of extrachromosomal nucleic acid elements.

The term “infection” as used hereinrefers to the transmission of a vector carried by a phage to a host cell by interaction of the phage with the host cell. In certain embodiments, infection comprises the phage attaching to the cell and inserting the vector into the cell.

The term “transformation,” as used herein, refers to the transmission of nucleic acid through a cell membrane and into a cell without the use of a transmitting agent. Transformation typically refers to the transmission of nucleic acid into bacterial cells.

The term “transfection,” as used herein, refers to the transmission of nucleic acid through a cell membrane and into a cell by means other than infection. Transfection typically refers to the transmission of nucleic acid into eukaryotic cells.

As used herein, the term “transduction” refers to the transmission of nucleic acid from one cell to another cell by a phage. In certain embodiments, during transduction, nucleic acid from a cell's genome is incorporated into a phage vector during phage assembly.

The term “conjugation,” as used herein, refers to the transmission of nucleic acid from one bacterial cell to another. Conjugation typically involves a nucleic acid encoding a fertility factor (F+). In certain embodiments, F+ sequences are found on a small supernumerary vector, such as an episome. Typically, when a cell containing an F+ (F+ cell) is in the presence of a cell not containing an F+ (an F− cell), the F+ cell grows a pillus, which then attaches to the F− cell. In certain embodiments, after attachment of the pillus, the F+ containing-nucleic acid is transmitted from the F+ cell to the F− cell, which results in the prior F− cell becoming an F+ cell.

Phage Display

It has been known to select binding moieties using “phage display.” “Phage display” refers to methods of selecting binding moieties by testing potential binding moieties presented on the surface of phage. In contrast to phage display, in the present invention, potential binding moieties are presented on a surface of the host cell or in an accessible compartment of the host cell. Phage display methods have been discussed, e.g., in de Bruin, et al., “Selection of High-Affinity Phage Antibodies from Phage Display Libraries,” Net. Biotechnol., 17:397-399 (1999); McCafferty, “Phage Display: Factors Affecting Panning Efficiency,” in Phage Display of Peptides and Proteins: A Laboratory Manual, Kay et al. eds., Academic Press, (1996); Bass, et al., “Hormone Phage: An Enrichment Method for Variant Proteins with Altered Binding Properties,” Proteins, 8:309-314 (1990); and Barbas, et al., “Assembly of Combinatorial Antibody Libraries On Phage Surfaces: The Gene III Site,” Proc. Nat. Acad. Sci. USA, 88:7978-7982 (1991).

Phage display methods of identification of binding moieties typically takes many days, according to some methods. A typical method of phage display comprises the following.

The first day, host cells are grown until they reach the mid-log phase of their growth. The phage stock is then titered in order to determine the concentration of the phage. The phage stock is serially diluted such that there are small amounts of phage at 1× concentration, 0.1× concentration, 0.01× concentration, and so forth. Each of these dilutions is then used to infect different small amounts of the host cells, which are then plated on agar and allowed to grow overnight in order to titer the phage. Plates with targets are prepared for use in selection.

On the second day, host cells are again grown for phage infection. The plates with targets are washed to remove excess targets. The phage stock is diluted appropriately after determining the phage titer, and diluted phage is added into separate wells of the plates with targets. After a period of incubation, allowing phage expressing binding moieties to bind to the targets bound to the plate, the nonbinding phage are poured off the plates, the plates are washed to remove the weakly bound phage, and the bound phage are eluted from the plates. The phage eluted from the plates are then titered by serially diluting the phage, adding the dilutions to logarithmically growing bacterial cells, plating the infected cells onto plates, and growing overnight as described previously. At the same time, the eluted phage are used to infect a second portion of logarithmically growing host cell culture for amplification. After several hours, the phage have infected the host cells and produced more phage, which in turn infect more host cells that produce more phage. After several hours, the phage have amplified, and are isolated and concentrated according to known methods.

On the third day, the isolation and concentration of the phage is completed. The concentrated phage is then titered by serial dilution and growing overnight on agar plates, as described previously. More plates with the target are prepared.

On the fourth day, the phage from the titer plates are counted, and the concentration of the phage is determined. The selection process with the target plates is then repeated. Eluted phage are then amplified by infecting host cell cultures as described above. More host cell cultures are started for subsequent use. Amplified phage are then titered overnight. On subsequent days, the process performed on day four is repeated.

The process for isolating a binding moiety by the phage display method may take several days of selection, amplification, and titering. In phage display methods, one typically titers the phage overnight every time phage is reamplified. This may make the entire process last for weeks.

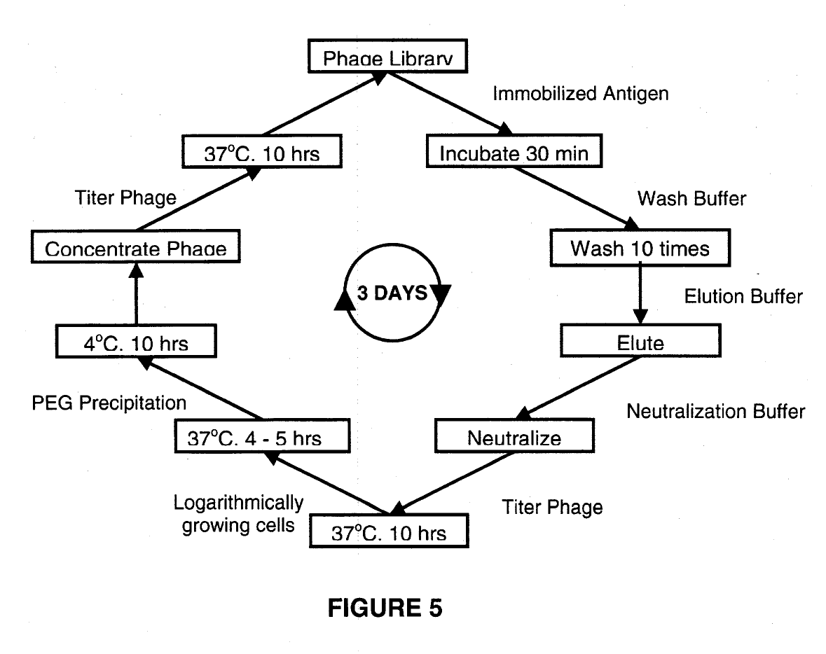

Repeated rounds of amplification and growth of phage allows for bias in the potential binding moieties isolated. Phage that result in slow growth of the host cell become under-represented during the selection process. Similarly, phage that result in rapid growth of the host cell become over-represented during the selection process. More prolonged the growth during the amplification, and more rounds of selection, increases the likelihood of bias in selection. A diagram of an exemplary cycle of selection in certain phage display techniques is illustrated in FIG. 5.

Also, with phage display techniques, the expression level of the potential binding moiety affects the efficiency of the selection method. The more potential binding moiety expressed on the surface, the greater the efficiency of enrichment for binding moieties in each successive selection step. Phage display techniques employing filamentous phage can employ different proteins fused to the potential binding moieties to display the moieties on the surface of the phage. Phage display using the gene III protein, for example, will typically express one to five copies of a protein on the phage surface. Some proteins may allow up to about 30 copies of a moiety to be displayed on the surface.

Typically, the efficiency of selection of a binding moiety in phage display is affected by the recovery of background phage that do not bind to targets. The use of large phage libraries increases the chance of selecting a specific binding moiety that binds to a target of interest. However, large libraries also increase the chance of obtaining nonspecifically-binding background phage that encode potential binding moieties that do not bind the target of interest. In addition, many phage may bind nonspecifically to surfaces of vessels and plates used in the selection process. The number of nonspecifically-binding background phage may reduce the efficiency of the selection of a binding moiety.

Initially, the starting phage may contain 1-10 binding moieties for every 10⁹ members of the phage pool. At the end of the many selection steps, there may be 1-10 binding moieties for every 10 members of the phage pool. Typically, there is a 10³-10⁵ fold enrichment of phage producing a binding moiety for each selection process. For example, following the initial binding, wash, and elution, the initial phage library may be reduced to 10⁴ to 10⁵ members of the library. Within these selected phage, there may be only 1 to 10 members that encode potential binding moieties that actually bind the target.

Also, because the phage expressing binding moieties must be eluted off the plates, many high affinity binding moieties never elute, and therefore do not get selected as binding moieties. In certain embodiments of the present invention, very high affinity binding moieties are not selected against.

Using certain phage display techniques, one titers phage, centrifuges cells and phage to separate cells from phage, and concentrates phage using PEG precipitation. In certain embodiments of the present invention, there is no need to titer phage. In certain embodiments of the present invention, there is no need to separate cells from phage by centrifugation. In certain embodiments of the present invention, there is no need to concentrate phage using PEG precipitation.

Phage Display Performed With the Target Expressed on Bacterial Surface

Some methods, such as delayed infectivity panning (DIP) employ phage display techniques. DIP has been discussed in Benhar et al., “Highly Efficient Selection of Phage Antibodies Mediated by Display of Antigen as Lpp-OmpA′ Fusions on Live Bacteria,” J. Mol. Biol. 301:893-904 (2000).

DIP employs the growth F+ bacterial host that produce target molecules on the cell surface. The host cells are rendered temporarily F− by growing the cells at 16° C. This prevents infection of the cells by phage.

Phage with potential binding moieties on their surfaces are exposed to bacterial cells expressing target molecules that are presented on the cell surface. The unbound phage are then removed from the cells.

The temperature of the bacterial environment is then raised to allow the bacteria to express an F+ pilus. This allows for infection by the phage. The phage are then unbound from the bacteria, and allowed to infect the bacterial cells. In this method, phage must still be tittered to determine concentration.

In this method, potential binding moieties are not presented on cell surfaces or in accessible compartments. The selection process in this method employs only potential binding moieties presented on phage.

Certain Exemplary Components and Methods

Introduction

In many instances, it may be desirable to identify moieties that specifically bind to particular targets. In certain embodiments, the process for identifying binding moieties that bind targets comprises the selection of a binding moiety from multiple potential binding moieties. In certain embodiments, the multiple potential binding moieties are in a library of potential binding moieties. As discussed below, exemplary sources of libraries are known to those of skill in the art. In various embodiments, many different types of libraries may be employed.

In certain embodiments, potential binding moieties are peptides encoded by nucleic acids. In certain embodiments, a nucleic acid encoding a potential binding moiety is part of a larger nucleic acid that encodes additional polypeptide. In such embodiments, the nucleic acid encoding the entire polypeptide, including the potential binding moiety portion of the polypeptide, is called a polypeptide encoding portion of a nucleic acid. In certain embodiments, the polypeptide encoding portion is part of a nucleic acid vector.

In certain embodiments, the process for identifying binding moieties that bind targets proceeds with a selection phase and a subsequent amplification phase as follows. In the following discussion, a host cell is discussed. In certain embodiments, of course, one employs multiple different potential binding moieties associated with different host cells. In certain such embodiments, one may determine whether any of the multiple different potential binding moieties bind to one or more targets.

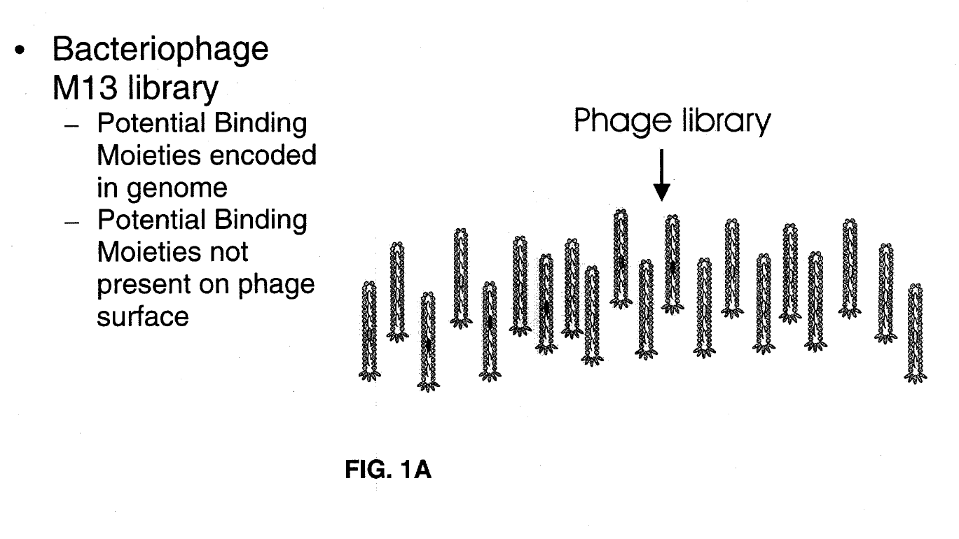

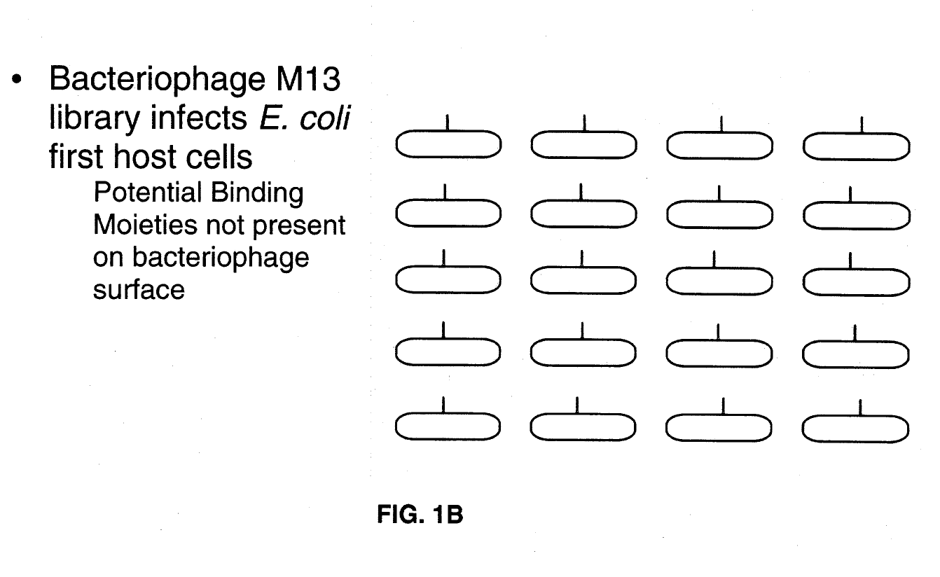

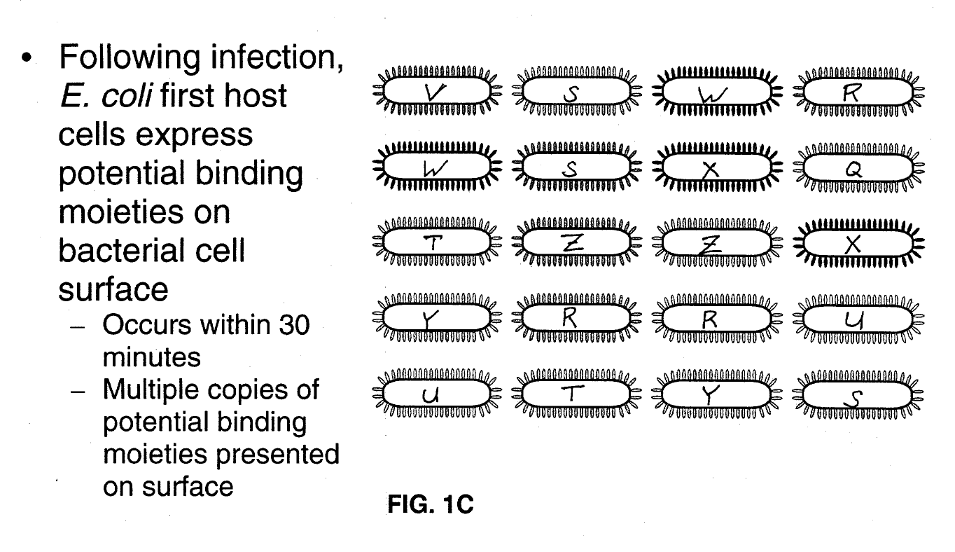

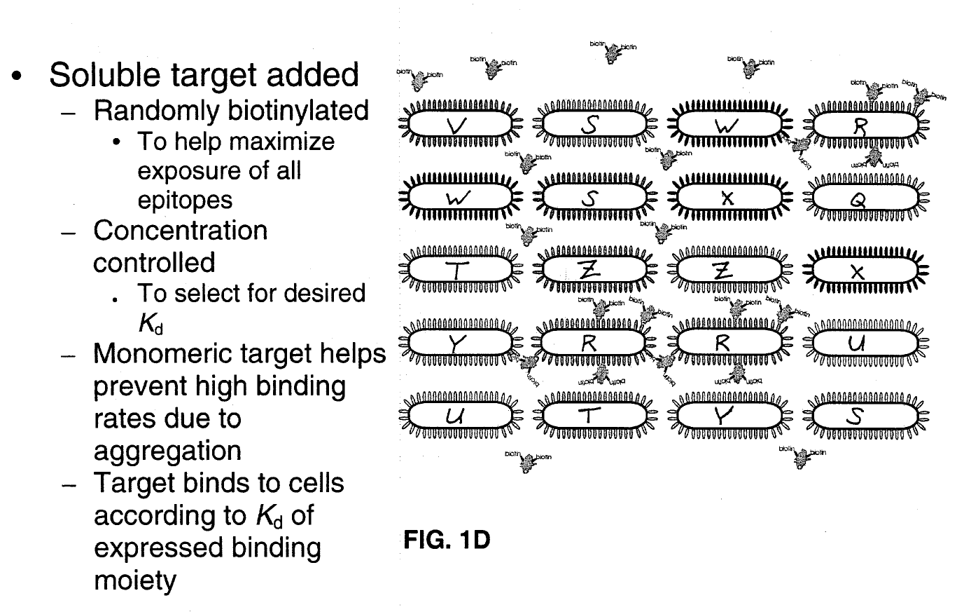

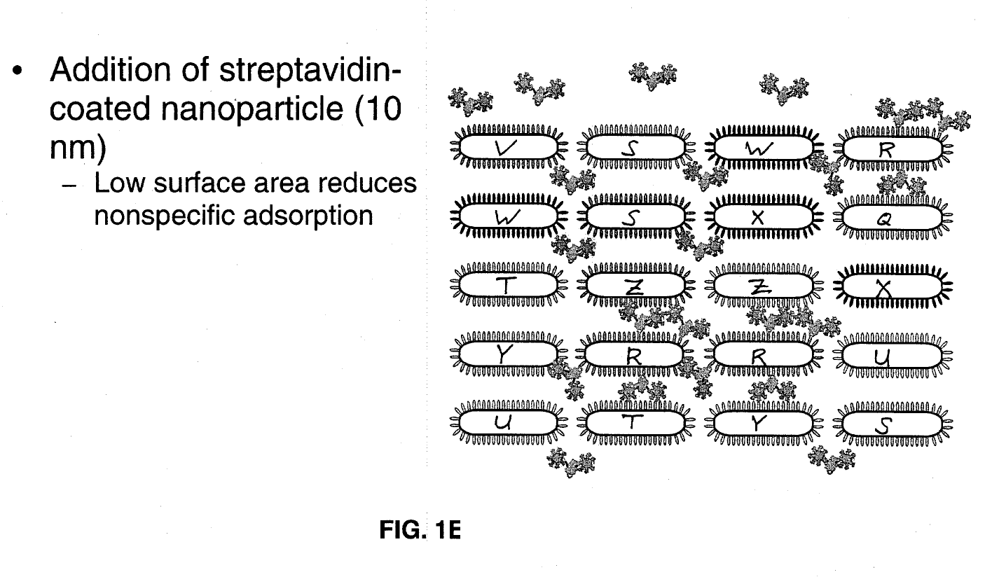

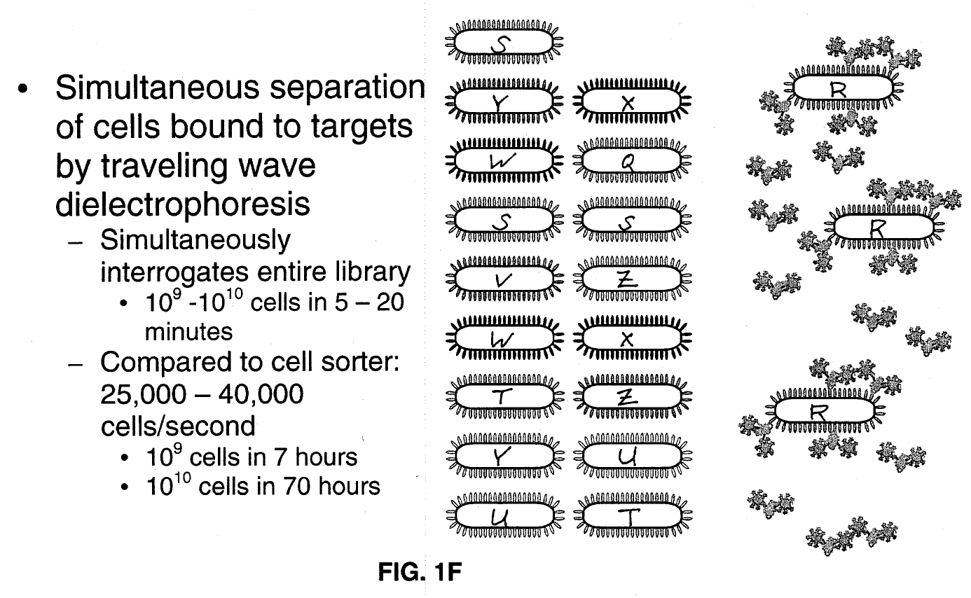

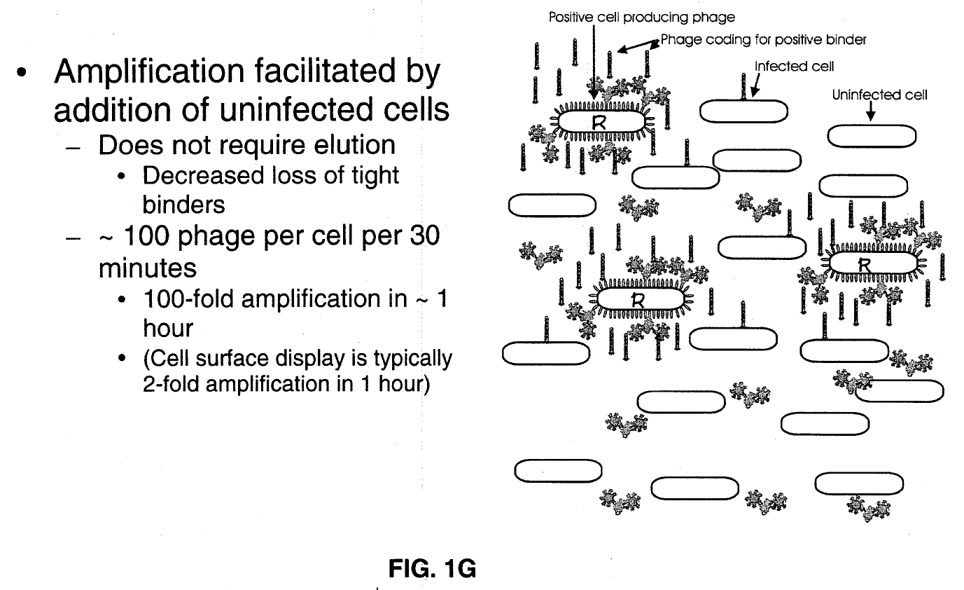

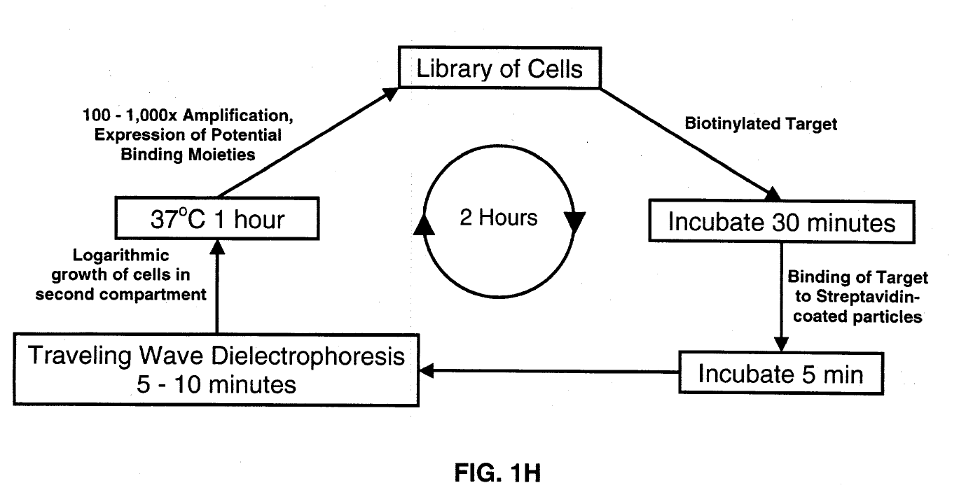

In certain embodiments, one employs a vector that comprises a nucleic acid that encodes a potential binding moiety. In certain embodiments, the vector is an M13 bacteriophage vector. See, e.g., FIG. 1A. In certain embodiments, for a selection phase, the vector is included in a first host cell. In certain embodiments, the vector is placed directly into the first host cell. Exemplary methods of placing a vector into first host cells include, but are not limited to, infection, transfection, transduction, conjugation, and transformation.

In certain embodiments, the nucleic acid in the vector encodes additional polypeptide comprising other elements for presenting the potential binding moiety. In certain embodiments, the polypeptide further comprises at least one of a start methionine, a secretory leader, a peptidase cleavage site, a site recognized for cleavage, a linker portion, and an anchor portion. In certain embodiments, the secretory leader of the polypeptide allows at least a portion of the polypeptide to pass through a membrane. In certain embodiments, a secretory leader contains a peptidase cleavage site, which may be cleaved by leader peptidase. In certain such embodiments, cleavage of the protein at the peptidase cleavage site allows for the removal of an epitope tag so that a protein without an epitope tag may be generated. In certain embodiments, an anchor portion of the polypeptide attaches the polypeptide to a membrane.

In certain embodiments, the vector further comprises other nucleic acid elements for regulation and transcription of the polypeptide encoding portion. In certain embodiments, the vector further comprises at least one of an operator, an initiator, a promoter, a transcription initiation sire, a ribosomal binding site, a start codon, a termination codon, and a transcriptional terminator. In certain embodiments, the vector further comprises elements that allow the vector to replicate. In certain embodiments, the vector further comprises elements that allow the transmission of the vector to a second host cell.

In certain embodiments, a potential binding moiety is produced inside the first host cell. In certain embodiments, the first host cell is E coli. See, e.g., FIGS. 1B and 1C. In certain embodiments, the potential binding moiety is presented in a manner such that it remains associated with the cell in which it is expressed so that it is associated with the nucleic acid encoding it. In certain embodiments, the potential binding moiety is also presented in a manner so that it is accessible for binding to a target.

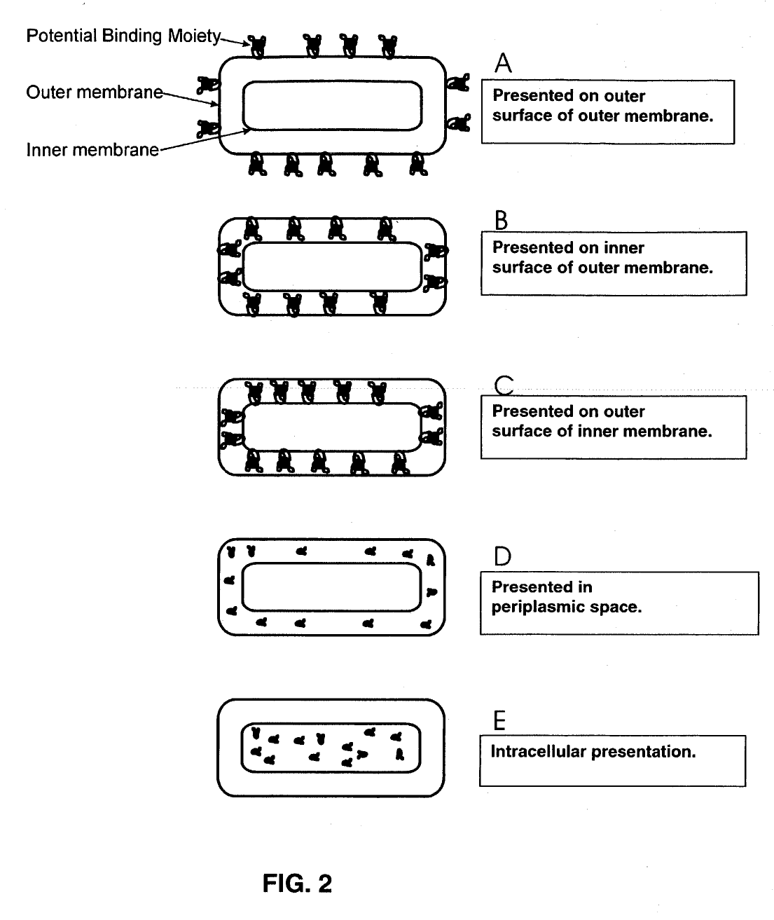

In certain embodiments, a potential binding moiety is presented in an accessible compartment of the first host cell. In certain embodiments, the potential binding moiety is presented on a surface of the first host cell. See, e.g., FIG. 2A. In certain embodiments, the potential binding moiety is presented on a surface of a membrane of the first host cell. In certain embodiments, the potential binding moiety is presented in the periplasmic space of a bacterial first host cell. In certain embodiments, the potential binding moiety is presented in an accessible compartment and is attached to an inner membrane of the host cell. An example of certain such embodiments is shown in FIG. 2C. In certain embodiments, the potential binding moiety is presented in an accessible compartment and is attached to an outer membrane of a first host cell. An example of certain such embodiments is shown in FIG. 2B. In certain embodiments, the potential binding moiety is not attached to a membrane and is presented in an accessible compartment within the first host cell. An example of certain such embodiments is shown in FIGS. 2D and E.

In certain embodiments, the first host cells with potential binding moieties on their surface or in an accessible compartment are exposed to targets. If a potential binding moiety has an affinity for the target, the potential binding moiety binds the target. A non-limiting example of a potential binding moiety binding a target is shown in FIG. 1D. In certain embodiments, such a target is then attached to other secondary labels, such as streptavidin-coated particles, as a non-limiting example, as shown in FIG. 1E. In certain embodiments, first host cells with potential binding moieties on their surface that are bound to targets are separated from first host cells that do not have potential binding moieties on their surface that are bound to targets. See, e.g., FIG. 1F, as a non-limiting example. In certain embodiments, first host cells with potential binding moieties in their accessible compartments that are bound to targets are separated from first host cells that do not have potential binding moieties in their accessible compartments that are bound to targets.

In certain embodiments, a subsequent amplification phase is employed. Amplification comprises replicating nucleic acid encoding a potential binding moiety, and transmitting the nucleic acid encoding the potential binding moiety to at least one second host cell that does not comprise a nucleic acid encoding the potential binding moiety. In certain embodiments, nucleic acid encoding the potential binding moieties in the selected first host cells replicate. See, e.g., FIG. 1G. In certain embodiments, the nucleic acids encoding the potential binding moieties are transmitted to second host cells. See, e.g., FIG. 1G. An exemplary cycle of potential binding moiety expression, separation, and amplification according to certain embodiments is shown FIG. 1H.

In certain embodiments, the second host cells are used to identify the potential binding moiety encoded by the nucleic acid. In certain embodiments, the second host cells are used to identify the nucleic acid encoding the potential binding moiety. In certain embodiments, the second host cells are used to produce the potential binding moiety. In certain embodiments, the potential binding moiety is identified by binding of the potential binding moiety to another moiety. In certain embodiments, the potential binding moiety is identified by hybridization of the nucleic acid encoding the potential binding moiety to nucleic acids complementary to known nucleic acid sequences. In certain embodiments, the known nucleic acid sequences are known potential binding moieties. In certain embodiments, the nucleic acids complementary to known nucleic acid sequences are attached to a substrate. In certain embodiments, the nucleic acids complementary to known nucleic acid sequences are attached to an array. In certain embodiments, the nucleic acids bound to the array are sequences complementary to potential binding moieties represented in a library of potential binding moieties. In certain embodiments, the potential binding moiety is identified by sequencing the nucleic acid encoding the potential binding moiety.

Exemplary Methods

Exemplary Selection Methods

In certain embodiments, host cells are selected when they express polypeptides comprising potential binding moieties that bind to targets. In certain embodiments, the polypeptides are presented in accessible compartments of the host cell. In certain embodiments, the polypeptides are presented on the surface of the host cell.

In certain embodiments, cell surfaces can display 10⁴-10⁵ copies of a binding moiety per cell. In certain embodiments, cell surfaces can display 10³-10⁴ copies of a binding moiety per cell. In certain embodiments, cell surfaces can display 10²-10³ copies of a binding moiety per cell. In certain embodiments, cell surfaces can display less than 100 copies of a binding moiety per cell. In certain embodiments, cell surfaces can display greater than 10⁵ copies of a binding moiety per cell.

In certain embodiments, the number of copies of a binding moiety expressed per cell can be regulated. In certain embodiments, one employs a vector comprising an inducible regulatory element. In certain embodiments, expression of a polypeptide comprising a potential biding moiety occurs when the vector is exposed to an inducer molecule or molecules or the cells are exposed to induction conditions, including, but not limited to, temperature and light. In certain such embodiments, one regulates the amount of an inducer molecule to which the vectors are exposed to regulate the amount of a potential binding moiety expressed. Examples of such inducer molecules and regulatory elements include, but are not limited to, use of arabinose for expression controlled by the araBAD element; use of galactose in conjunction with the gal E promoter element; use of tryptophan in conjunction with the trp E promoter element; use of maltose in conjunction with the mal T, mal E, mal K, mal S, or mal I promoter elements; and use of Isopropyl-β-D-thiogalactoside (IPTG) in conjunction with a LacZ promoter element. In certain embodiments, variability in expression levels or variability instability among different potential binding moieties expressed does not substantially affect selection of the potential binding moiety.

In certain embodiments, selection methods comprise exposing cells with potential binding moieties on their surfaces to targets. In certain embodiments, targets are bound by potential binding moieties that have a sufficient affinity for the targets. In certain embodiments, the concentration of the target affects the affinity of the potential binding moiety that is selected. In certain embodiments, employing a lower concentration of target selects higher affinity binding moieties.

In certain embodiments, targets are labeled with a label. In certain embodiments, the target may be directly attached to a label that distinguishes cells with potential binding moieties bound to targets from cells that do not have potential binding moieties bound to targets. Several methods of directly attaching labels to targets are known to one of skill in the art.

In certain embodiments, the target is indirectly attached to another moiety that distinguishes cells with potential binding moieties bound to targets from cells that do not have potential binding moieties bound to targets. In certain embodiments, the target is attached to a first label that binds to a second label, such as a member of an affinity binding set. In certain embodiments, the second label is attached to a third label that distinguishes cells with potential binding moieties bound to targets from cells that do not have potential binding moieties bound to targets. For example, in certain embodiments, a target may be attached to biotin. After the target is bound to a potential binding moiety, the target is exposed to magnetic particles attached to streptavidin. The streptavidin binds the biotin, and the magnetic particles are used to distinguish cells with potential binding moieties bound to targets from cells that do not have potential binding moieties bound to targets.

Another non-limiting example of indirect attachment employs four labels. For example, in certain embodiments, an unlabeled target may bind a potential binding moiety. An antibody that recognizes the target is attached to biotin. The antibody binds to the target that is bound to the potential binding moiety. After the antibody is bound to the target, the antibody is exposed to magnetic particles attached to streptavidin. The streptavidin binds the biotin, and the magnetic particles are used to distinguish cells with potential binding moieties bound to targets from cells that do not have potential binding moieties bound to targets.

In certain embodiments, a target is soluble, and is exposed to first host cells with potential binding moieties in solution. In certain embodiments, the soluble target is biotinylated, and may be captured on a solid substrate coated with streptavidin, either before or after binding by a binding moiety. In certain embodiments, the target bound to a binding moiety may be labeled, and the cells bound to target may be separated in view of the label. In certain embodiments, the target is labeled before the cells are exposed to it.

In certain embodiments, cells that produce binding moieties capable of binding a target are enriched. In certain embodiments, the enrichment of cells that produce binding moieties capable of binding a target comprises: exposing the cells that produce binding moieties capable of binding a target to targets and subjecting the cells to at least one method of separation.

In certain embodiments, cells that produce binding moieties capable of binding a target are selected. In certain embodiments, selected cells with binding moieties bound to targets are separated from cells that do not have binding moieties bound to targets. In certain embodiments, separation is accomplished by a method that distinguishes cells that have a bound target from those that have no bound target. Non-limiting examples of such methods include, but are not limited to, traveling wave dielectrophoresis, dielectrophoresis, electrophoresis, diamagnetism, dialysis, sedimentation, centrifugation, magnetic separation, chromatography, and fluorescence-activated cell sorting.

Dielectropheretic separation of cells has been described, for example, in Rousselet, et al., 1998, “Separation of Erythrocytes and Latex Beads by Dielectrophoretic Levitation and Hyperlayer Field-Flow Fraction,” Colloids and Surfaces 140:209-216; Talary, et al., 1996, “Electromanipulation and Separation of Cells Using Travelling Electric Fields,” J. Phys. D.: Appl. Phys. 29:2198-2203; and Markx, et al., 1995, “Dielectrophoretic Separation of Cells: Continuous Separation,” Biotechnology and Bioengineering, 45:337-343. In certain embodiments, traveling wave dielectrophoresis may separate cell types possessing different dielectrophoretic properties to greater than 99% accuracy.

In certain embodiments, soluble target may be attached to a dielectrophoretic label, such as a latex bead or a gold particle as non-limiting examples. In certain embodiments, if a target with a dielectrophoretic label is bound to a cell, then the cell may be separated from cells without bound target based on its dieletrophoretic properties. In certain embodiments, the target is attached to a member of an affinity binding set, such as biotin. In certain embodiments, the dielectrophoretic label is coated with streptavidin. In certain embodiments, the dielectrophoretic labels are streptavidin-coated 10 nm particles sold by Polysciences, Inc. (Warrington, Pa.). In such embodiments, cells with biotinylated targets bound to potential binding moieties would be attached to gold particles that impart different dielectrophoretic properties compared to cells not attached to gold particles.

In certain embodiments, the binding of target to a potential binding moiety of a cell changes the physiology of the cell such that the dielectrophoretic properties of the cell changes. For example, in certain embodiments, the binding of the target to the binding moiety may change the cell permeability, resulting in an altered impedance of the cell. In certain embodiments, a target bound to a binding moiety may change the nucleus:cytoplasm ratio of the cell, resulting in a different dielectrophoretic property of that cell compared to cells in which no target is bound to a binding moiety.

In certain embodiments, magnetic fields may be used to separate cells bound to targets. In certain embodiments, targets are attached to a magnetic or paramagnetic label. In certain embodiments, the target is attached to a member of an affinity binding set, such as biotin, and the magnetic or paramagnetic label is coated with streptavidin. In certain embodiments, a magnetic field may then be applied to separate and isolate cells bound to targets with a magnetic label attached. In certain embodiments, if the target is attached to a biotin molecule, magnetic particles coated with streptavidin may be added to the cells prior to separation with the magnetic field.

In certain embodiments, target may be attached to a diamagnetic label such as copper, bismuth, lead, gallium. In certain embodiments, target may be attached to a paramagnetic label, such as various salts, metals, and nickel and cobalt at elevated temperatures. In certain embodiments, if a target with a diamagnetic or paramagnetic label is bound to a cell, then the cell may be separated from cells without bound target based on its diamagnetic or paramagnetic properties. In certain embodiments, the target is attached to a member of an affinity binding set, such as biotin. In certain embodiments, the label is coated with streptavidin.

In certain embodiments, electrophoresis may be used to separate cells bound to targets. In certain embodiments, targets are attached to labels that confer a charge or mobility modification. Exemplary labels used in electrophoretic separation methods include, but are not limited to, charged molecules, such as metallic salts and organic anions, and mobility modifiers, such as polyethylene glycol. In certain embodiments, when the cells have binding moieties that bind to such targets, the cells' electrophoretic mobility is changed. Such a change allows the separation of cells with binding moieties bound to labeled targets. In certain embodiments, the target is attached to a member of an affinity binding set, such as biotin. In certain embodiments, the electrophoretic label is attached to streptavidin.

Fluorescence-activated cell sorting has been described, for example, in Fuchs, et al., 1991, Targeting Recombinant Antibodies to the Surface of Escherichia coli: Fusion to a Peptidoglycan Associated Lipoprotein,” Biotechnology, 9:1369-1372; Daugherty, et al., 2000, “Flow Cytometric Screening of Cell-Based Libraries,” J. Immunol. Methods, 243:211-227; Daugherty et al., 1998, “Antibody Affinity Maturation Using Bacterial Surface Display,” Protein Engineering, 11:825-832; and Daugherty et al., 1999, “Development of an Optimized Expression System for the Screening of Antibody Libraries Displayed on the Escherichia coli Surface,” Protein Engineering, 12:613-621. In certain embodiments, a desired concentration of fluorescently-labeled target may be added to cells expressing a potential binding moiety. In certain embodiments, following incubation with the fluorescently-labeled target, fluorescence-activated cell sorting (FACS) may be used to sort and isolate cells bound to fluorescently-labeled target. In certain embodiments, the target is attached to a member of an affinity binding set, such as biotin. In certain embodiments, the fluorescent label is attached to another member of the affinity binding set, such as streptavidin.

In certain embodiments, the area or vessel in which selection occurs comprises one or more compartments. In certain embodiments, cells with potential binding moieties bound to targets are moved to one compartment to separate them from cells with potential binding moieties that are not bound to targets. In certain embodiments, cells that do not have potential binding moieties bound to targets are moved to one compartment to separate them from cells with potential binding moieties that are bound to targets. In certain embodiments, cells with potential binding moieties bound to targets are moved to a first compartment and cells that do not have potential binding moieties bound to targets are moved to a second compartment. In certain embodiments, the moving the cells with potential binding moieties bound to targets to a first compartment occurs simultaneously with the moving the cells that do not have potential binding moieties bound to targets to a second compartment. In certain embodiments, cells with potential binding moieties bound to targets are moved to a compartment comprising cells that do not comprise nucleic acid encoding a potential binding moiety. In certain embodiments, cells that do not comprise nucleic acid encoding a potential binding moiety are added to a compartment comprising cells with potential binding moieties bound to targets.

In certain embodiments, multiple different targets may be used together to identify multiple different binding moieties. In certain such embodiments, different targets have different labels. In certain embodiments, the different labels are detectably different. In certain embodiments, the different labels are different colored fluorescent molecules. In certain embodiments, cells with binding moieties bound to different targets are distinguishable by the different colored fluorescent molecules. In certain embodiments, the cells may be sorted by FACS. In certain embodiments, the different labels are different particles with different dielectrophoretic properties. In certain embodiments, cells with binding moieties bound to different targets are distinguished by the different dielectrophoretic particles. In certain embodiments, the cells may be sorted by traveling wave dielectrophoresis gradients.

In certain embodiments, identifying multiple different binding moieties employs nucleic acid labels. In certain such embodiments, nucleic acid labels may be attached directly to targets, or to a member of an affinity binding set, such as avidin that can bind a biotin molecule attached to a target. In certain embodiments, nucleic acid labels are then be detected by hybridization of complementary nucleic acids attached to fluorescent labels. In certain embodiments, nucleic acid labels are detected by nucleic acid detection methods including, but not limited to, PCR, oligonucleotide ligation assay (OLA), OLA-PCR, methods employing Taqman™ probes, and methods employing intercalating dyes.

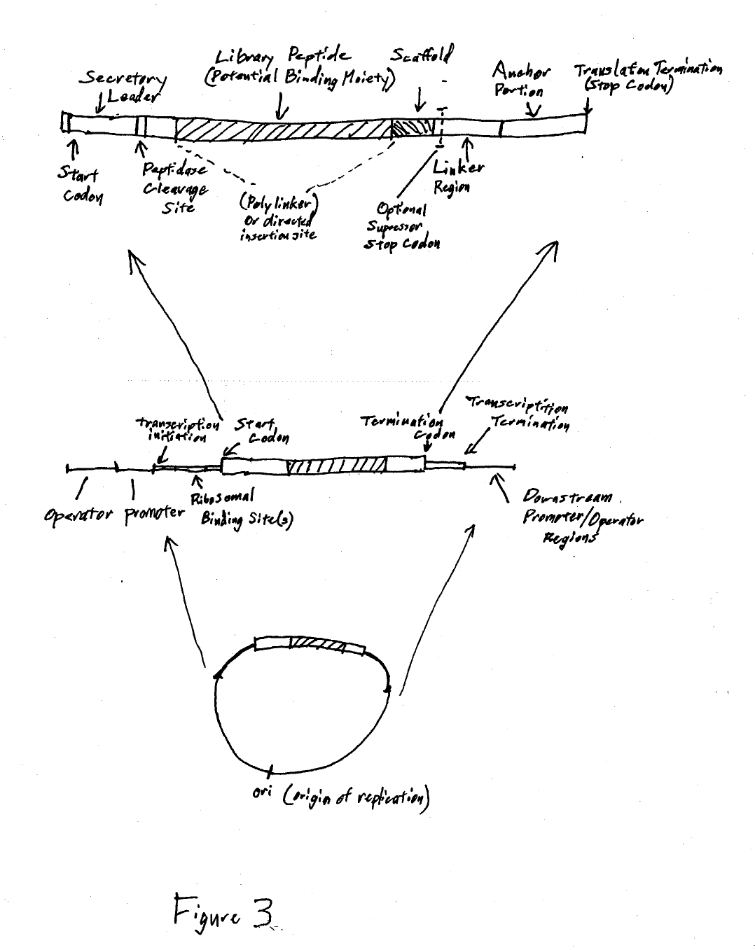

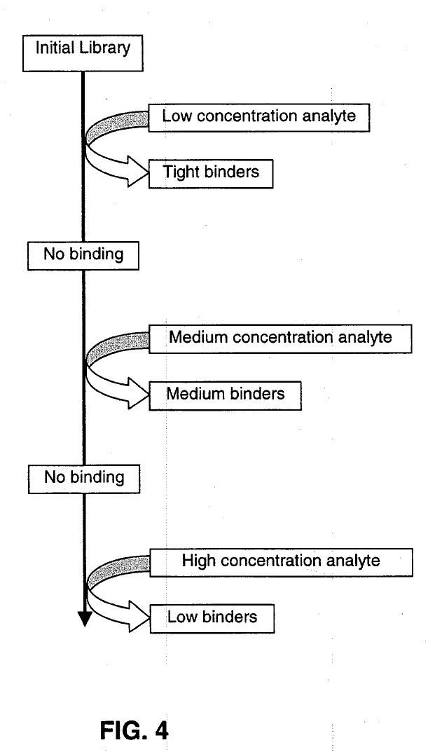

In certain embodiments, unlabeled targets may be used to select higher affinity binding moieties. In certain embodiments, after an initial selection and separation of cells expressing binding moieties, selected cells are subsequently incubated with high concentrations of targets that are not labeled. In certain embodiments, low affinity binding moieties release the labeled targets and bind unlabeled targets. In certain embodiments, the selected cells are then separated based on the presence of a labeled target bound to the cell. In certain embodiments, a specific affinity of binding moiety may be selected for by controlling the concentration of the target. See, e.g., FIG. 4, illustrating certain exemplary methods of selecting binding moieties of different affinities according to certain embodiments.

In certain embodiments, the amount of target used for selection is substantially less than the amount typically used in phage display techniques. In certain embodiments, for selection, 5 ng of a 50 kD protein is used for a 1 nM target concentration in a 100 μl volume. In certain embodiments, for selection, 1 μg of a 100 kD protein is used for a 100 nM target concentration in a 100 μl volume. In certain embodiments, for selection, 1 ng of a 100 kD protein is used for a 100 pM target concentration in a 100 μl volume.

In certain embodiments, vectors comprising nucleic acid sequences encoding a potential binding moiety are included in phage in a cell. In certain instances, phage that contain polypeptides comprising a potential binding moiety on the surface of the phage may compete with host cells for binding to targets during the selection process. In certain embodiments, potential binding moieties are not presented on the surface of phage, but are presented on the surface of a membrane of cells, or in an accessible compartment of the cells. In certain embodiments, there are substantially no phage present during selection that compete with host cells for binding to the target.

When using phage display techniques, phage are typically eluted from a solid medium. Phage encoding very high affinity binding moieties may not be released under typical elution conditions. In such conditions, those very high affinity binding moieties may not be selected. The conditions for the elution of phage in phage display techniques may denature potential binding moieties or denature targets. In certain embodiments, denaturation of potential binding moieties and targets is avoided by the present invention. In certain embodiments, no elution step is used to remove binding moieties attached to targets.

In some selection methods, potential binding moieties may be selected that do not have a specific affinity for the target. In certain embodiments, it may be desirable to reduce or eliminate such non-specific binding. As a non-limiting example, a target is attached to a biotin molecule which interacts with a streptavidin-coated surface. If some potential binding moieties bind streptavidin, then these potential binding moieties may be selected even though they are not bound to the target. In certain embodiments, unlabeled targets or other proteins may be used to increase the selectivity in such an instance. As a non-limiting example, in certain embodiments, if a streptavidin-coated solid surface is used to bind to targets to select for cells expressing binding moieties, a soluble streptavidin molecule that lacks a biotin-binding site can be used to reduce or prevent the selection of streptavidin-binding moieties expressed by the cells. The soluble streptavidin binds to potential binding moieties that bind streptavidin, which in turn blocks the potential binding moiety from binding to the streptavdin-coated surface used to select binding moieties that bind targets.

Also, in certain instances, selection of binding moieties that specifically bind phosphorylated proteins may be desired. In such instances, potential binding moieties that bind nonspecifically to an unphosphorylated form of the protein are undesirable. In certain embodiments, one may expose cells to soluble unphosphorylated proteins to bind such undesirable potential binding moieties to increase the selectivity for binding moieties that have an affinity for the phosphorylated region of a protein.

As another non-limiting example, in certain embodiments, it may be desirable to select potential binding moieties that bind to proteins expressed by cancerous cells, such as cancerous liver cells. In certain embodiments, one may wish to reduce or eliminate the selection of potential binding moieties that bind to many types of cells (such as normal liver cells, for example) other than the cancerous cells. In certain embodiments, cells expressing potential binding moieties may be exposed to a certain non-cancerous cell type, such as normal liver cells, for example. Cells expressing potential binding moieties that bind to the non-cancerous liver cells are removed. Then, the remaining cells expressing potential binding moieties are exposed to cancerous cells derived from the non-cancerous cell type, such as cancerous liver cells derived from the normal liver cells. In certain embodiments, this reduces or eliminates the selection of cells expressing potential binding moieties that generally bind to cell-specific molecules (such as liver cell specific proteins) that are not associated with a cell's cancerous state.

In certain embodiments, one labels the cancerous cell with biotin. The biotin-labeled cancerous cell is then included with unmodified normal cells. Host cells expressing potential binding moieties that only bind the cancerous cell, but not the normal cells, are bound to the cancerous cell. The unmodified normal cells bind to other host cells expressing potential binding moieties that bind nonspecifically, which eliminates or reduces the number of such undesirable cells that bind to cancerous cells. In certain embodiments, streptavidin-coated particles may then be added and used to separate the cancerous cells bound to host cells expressing binding moieties from cells not bound to cancerous cells. Certain non-limiting exemplary separation methods are discussed above, e.g., magnetism or traveling wave dielectrophoresis. In certain embodiments, the cancerous cell may have dielectrophoretic properties significantly different from the non-cancerous cell. In certain embodiments, one separates cancerous cells bound to host cells expressing potential binding moieties that only bind cancerous cells from normal cells using traveling wave dieletrophoresis.

In certain embodiments, cells expressing binding moieties occurring as rarely as one in 10⁷ cells are selected by methods of the present invention. In certain embodiments, cells expressing binding moieties occurring as rarely as one in 10⁶ cells are selected by methods of the present invention. In certain embodiments, cells expressing binding moieties occurring as rarely as one in 10⁵ cells are selected by methods of the present invention. In certain embodiments, cells expressing binding moieties occurring as rarely as one in 10⁴ cells are selected by methods of the present invention. In certain embodiments, cells expressing binding moieties occurring as rarely as one in 10³ cells are selected by methods of the present invention.

Amplification

In certain embodiments, after selection and separation of the cells expressing binding moieties, the vectors encoding the binding moieties are amplified. Amplification comprises replicating the nucleic acid encoding a potential binding moiety and transmitting the nucleic acid encoding the potential binding moiety to at least one second host cell that does not comprise nucleic acid encoding the potential binding moiety. In certain embodiments, amplification of the vector encoding the binding moiety enables identification of the binding moiety. Exemplary vectors comprising nucleic acid encoding the potential binding moieties include, but are not limited to, plasmids, cosmids, episomes, other extrachromosomal elements, and phage vectors.

In certain embodiments, replication of nucleic acid encoding a potential binding moiety occurs in at least one first host cell. In certain embodiments, replication of nucleic acid encoding a potential binding moiety occurs in at least one second host cell. In such embodiments, replication of nucleic acid encoding a potential binding moiety occurs after transmission of the nucleic acid encoding a potential binding moiety to the at least one second host cell.

In certain embodiments, replication of nucleic acid encoding a potential binding moiety employs enzymes and replicating activity of first host cells. In certain embodiments, nucleic acids in plasmids, cosmids, episomes, and other extrachromosomal elements comprising nucleic acid encoding a potential binding moiety are amplified in a manner that does not require cell division. Exemplary vectors that may comprise such a nucleic acid include, but are not limited to, plasmids, cosmids, episomes, F′ episomes, and other extrachromosomal elements.

In certain embodiments, transmission of nucleic acids encoding a potential binding moiety occurs by transfection. In certain embodiments, transmission of nucleic acids encoding a potential binding moiety occurs by transduction. In certain embodiments, transmission of nucleic acids encoding a potential binding moiety occurs by transformation. In certain embodiments, first host cells possessing vectors comprising nucleic acids encoding a potential binding moiety are lysed and the vectors are absorbed by second host cells. In certain embodiments, transmission of nucleic acids encoding a potential binding moiety occurs by electroporation. In certain embodiments, the nucleic acids encoding a potential binding moiety are transmitted by conjugation. In certain embodiments, a first host cell produces a pillus that allows the transmission of nucleic acids encoding a potential binding moiety from a first host cell to a second host cell.

In certain embodiments, vectors possess elements that allow replication of vectors. Exemplary elements that allow replication include, but are not limited to, an origin of replication, nucleic acid that encodes polymerases, and nucleic acids that encode ribosomes. In certain embodiments, the vector possess elements that allow the transmission of vectors to a second host cell. Exemplary elements that allow the transmission of vectors to a second host cell include, but are not limited to, viral coat proteins, F+ factors, and enzymes used for phage assembly. In certain embodiments, transmission of vectors to a second host cell employs a transmission agent. In certain embodiments, the transmission agent is a phage.

In certain embodiments, amplification of a vector occurs by phage replication and assembly. In certain embodiments, replication of a vector occurs by phage replication. In certain embodiments, replication of vectors comprising nucleic acid encoding the potential binding moiety that are in phage enables large scale production of the nucleic acid encoding the potential binding moiety.

In certain embodiments, vectors from a non-lytic phage are used to express potential binding moieties in cells. In certain such embodiments, a cell will simultaneously express potential binding moieties and produce phage that will be secreted into the medium in which the cell is growing. In certain embodiments, the secreted phage will be able to infect uninfected cells.

In certain embodiments, amplification is accomplished by exposing second host cells, that do not comprise nucleic acids encoding the potential binding moieties, to a selected at least one first host cell; and transmitting the nucleic acid encoding the potential binding moiety to at least one second host cell. In certain embodiments, the vector comprising the nucleic acid encoding the potential binding moiety replicates, produces phage proteins, and phage are assembled. In certain embodiments, the phage cause the cell to lyse and the phage are able to infect second host cells. In certain embodiments, the phage infect second host cells, and in turn more vectors are replicated and more phage are assembled. In certain embodiments, the phage lyse the second host cells, and are able to infect third host cells. In certain embodiments, amplification may involve one or more additional rounds of phage production and phage infection.

In certain embodiments, a host cell produces approximately 100-200 phage after infection. In certain embodiments, 200-1,000 phage are produced by a host cell following infection. In certain embodiments, less than 100 phage are produced by each host cell following infection. In certain embodiments, greater than 1,000 phage are produced by each host cell following infection. In certain embodiments, five initial phage may infect cells and replicate more phage until titers of 10¹² phage per ml are reached within three hours.

In certain embodiments, selected cells expressing binding moieties are mixed with an excess of uninfected host cells. In certain embodiments, infected cells produce 100-1,000 phage per round of phage production and infection in 30 to 40 minutes. In certain embodiments, a 100 to 1,000 fold replication of the initial phage is achieved in less than one hour. In certain embodiments, selected cells expressing binding moieties are diluted into a culture of uninfected host cells. In certain embodiments, the number of cells are monitored spectrophotometrically. In certain embodiments, the host cells possess a fluorescent indicator (e.g., green fluorescent protein), and the number of cells are monitored spectrofluorometrically.

In certain embodiments, an infection cycle comprises the infection of a cell by a phage, the replication of the phage vector and assembly of new phage, and the lysis of the infected cell, releasing the new phage. In certain embodiments, the amplification is accomplished through one or two infection cycles and the entire amplification process lasts 1 to 1.5 hours. In certain embodiments, one infection cycle produces at least 100 fold more phage than the initial number of phage in 30 to 40 minutes.

In certain embodiments, amplification of vectors encoding binding moieties may be followed by a second selection and separation process. In certain embodiments, the second selection and separation process may be followed by a second amplification process. In certain embodiments, several successive rounds of selection and separation, followed by amplification, may be used. In certain embodiments, binding moieties may be selected and generated in two days.

In certain embodiments, replication is performed to produce multiple copies of a vector for identification of the nucleic acid. In certain embodiments, vectors are isolated from phage or cells, and the nucleic acid encoding the binding moiety is sequenced. In certain embodiments, the sequencing of the nucleic acid encoding the binding moiety identifies the binding moiety. In certain embodiments, amplification results in multiple newly infected cells expressing the binding moiety. In certain embodiments, newly infected cells produce soluble polypeptides comprising binding moieties and the binding moieties are isolated from the phage and cells and purified.

In certain embodiments, the second host cells are used to identify the potential binding moiety encoded by the nucleic acid. In certain embodiments, the second host cells are used to identify the nucleic acid encoding the potential binding moiety. In certain embodiments, the second host cells are used to produce the potential binding moiety. In certain embodiments, the potential binding moiety is identified by binding of the potential binding moiety to another moiety. The use of different host cells to perform different functions such as amplification, selection, and identification has been described, for example, in Zhao, Kong-Nan et al., “Saccharomyces cerevisiae is Permissive for Replication of Bovine Papillomavirus Type 1,” J. of Virology, 76(23):12265-12273 (2002).

In certain embodiments, the potential binding moiety is identified by hybridization of the nucleic acid encoding the potential binding moiety to nucleic acids complementary to known nucleic acid sequences. In certain embodiments, the known nucleic acid sequences are known potential binding moieties. In certain embodiments, the nucleic acids complementary to known nucleic acid sequences are attached to a substrate. In certain embodiments, the nucleic acids complementary to known nucleic acid sequences are attached to an array. In certain embodiments, the nucleic acids bound to the array are sequences complementary to potential binding moieties represented in a library of potential binding moieties. In certain embodiments, the potential binding moiety is identified by sequencing the nucleic acid encoding the potential binding moiety.

Certain Exemplary Components

Certain Exemplary Libraries and Potential Binding Moieties

In certain embodiments, a library of nucleic acid sequences is used to select one or more binding moieties. In certain embodiments, a library includes a set of different sequences encoding different potential binding moieties. Exemplary potential binding moieties include, but are not limited to, antibodies, single-chain antibodies, F_{ab} fragments, single domain antibodies, an antibody library from a nonimmunized spleen, constrained peptides such as cyclic peptides with cysteines permitting disulfide bond formation, and scaffold proteins containing randomized peptide sequences. In certain embodiments, the potential binding moieties are receptor sequences, other binding protein sequences, and/or enzyme sequences. In certain embodiments, the potential binding moieties are peptides that have been modified or randomly mutated to generate proteins with binding characteristics different from the peptides from which they have been modified.

In certain embodiments, libraries may represent peptides expressed in different cell types. As a non-limiting example, a library may be created from mRNA expressed in spleen cells. In such an embodiments, cDNA may be made from the mRNA, and the cDNA is used to create a library of nucleic acids encoding spleen cell proteins. In certain embodiments, a library may be a collection of nucleic acids encoding random peptides. In certain embodiments, random peptides may also be modified to favor the inclusion of certain amino acid residues, or peptides of certain lengths. In certain embodiments, libraries of nucleic acids are included in vectors, wherein all the vectors are identical with the exception of the nucleic acid library members. In certain embodiments, members of a library are each found in a separate vector. In certain embodiments, the members of the library are not present in vectors. In certain embodiments, the members of a library may be easily moved from one vector to another vector using standard molecular biology techniques.

Exemplary libraries are available commercially as cDNA, and include, but are not limited to cDNAs from human, mouse, mouse brain, rat, Drosophila, Xenopus, Zebrafish, human tumor, human fetal, plants, human adipose tissue, human breast, human colon, human heart, human hippocampus, human kidney, human liver, human lung, human skeletal muscle, human ovary, human placenta, human prostate, human skin, human spleen, human pancreas, human medulla, human brain tumor, human breast tumor, human colon tumor, human hippocampus tumor, human fetal spinal chord, human stomach, human Alzheimer's brain, Drosophila embryo, mouse embryo, Hela cells, K562 cells, Rabbit Bone Marrow, and peripheral blood. Commercial cDNA libraries are available from, for example, Invitrogen (Carlsbad, Calif.) and Novagen (Madison, Wis.).

In certain embodiments, a cDNA library may be created from the RNA of any tissue using standard molecular biology techniques, such as those described in Current Protocols in Molecular Biology, for example. The cDNA library may represent the entire population of expressed messenger RNA. Alternatively, mRNA may be expressed differently in one cell type under certain conditions may be used to create the library of potential binding moieties.

Certain Exemplary Polypeptide Construction

In certain embodiments, a vector comprises a nucleic acid sequence that encodes a polypeptide comprising a potential binding moiety. In certain embodiments, the polypeptide encoded by the vector has an N-terminal methionine or valine.

In certain embodiments, a vector comprises at least one nucleic acid sequence that encodes a polypeptide that makes a carbohydrate or polysaccharide, or attaches a carbohydrate or polysaccharide to a protein. In certain embodiments, a vector comprises at least one nucleic acid that encodes a polypeptide that produces an organic chemical molecule. For example, in certain embodiments, a polypeptide may be an enzyme or other moiety involved in the production of at least one of a carbohydrate, polysaccharide, and organic chemical molecule.

In certain embodiments, the nucleic acid encoding the polypeptide comprises nucleic acid encoding a carrier protein that results in the polypeptide being presented on the surface of a cell. In certain embodiments, the nucleic acid encoding the polypeptide comprises two nucleic acids encoding carrier proteins. In certain embodiments, the carrier protein results in the polypeptide being presented in an accessible compartment of a cell. In certain embodiments, the carrier protein will allow the potential binding moiety to be presented on the surface of the cell, or in an accessible compartment, but not on the surface of a phage. Exemplary carrier proteins include, but are not limited to, maltose-binding protein; LamB; petidoglycan associated protein; outer membrane protein; Lpp-OmpA; Pseudomonas aeruginosa major lipoprotein (Oprl); Pseudomonas OrpF; phoE; ompA; ice nucleation protein; F pillin; an autotransporter protein; Shigella VirG_{β}; Neisseria IgA_{β}; Flagellin; and fibriae.

In certain embodiments, the polypeptide comprises a secretory leader. In certain embodiments, the polypeptide comprises two secretory leaders. In certain embodiments, the polypeptide comprises two secretory leaders and two carrier proteins. In certain embodiments, the secretory leader is adjacent to the starting amino acid. In certain embodiments, the peptide further comprises a cleavage site after the secretory leader which permits cleavage of the secretory leader from the mature polypeptide. In certain embodiments, the secretory leader is internal within the polypeptide. In certain embodiments, polypeptides with a secretory leader pass at least partially through the inner bacterial membrane into the periplasmic space. In certain embodiments, polypeptides with a secretory leader pass at least partially through the outer bacterial membrane. In certain embodiments, the secretory leader anchors the peptide to the inner bacterial membrane. In certain embodiments, the secretory leader anchors the peptide to the outer bacterial membrane.

In certain embodiments, the cleavage site is a leader peptidase sequence. In certain embodiments, a polypeptide lacking a cleavage site attaches at the amino terminal end to the inner bacterial membrane, keeping the polypeptide in the periplasmic space.

In certain embodiments, the polypeptide further comprises a scaffold. A scaffold is a peptide that provides some secondary structure within a polypeptide. In certain embodiments, providing a specific secondary structure to a polypeptide may facilitate a potential binding moiety binding a target. Exemplary scaffolds include, but are not limited to, randomly derivatized peptide loops, Trinectin, portions of fibronectin, anticallin, thioredoxin, human placental ribonuclease inhibitor, antibodies, antibody heavy chains, antibody light chains, and single-domain antibodies.

In certain embodiments, the polypeptide comprises an anchor portion. In certain embodiments, the anchor portion attaches the polypeptide to the inner membrane of a host cell and a portion of the polypeptide is exposed to the inner surface of the periplasmic space. In certain embodiments, an anchor portion is a peptide that is substantially incapable of being transported across a membrane. In certain embodiments, when a portion of a polypeptide with an anchor portion is transported across a membrane, the anchor portion does not pass through the membrane, and the polypeptide becomes attached to the membrane. In certain embodiments, the anchor portion attaches the polypeptide to the outer membrane and exposes a portion of the polypeptide to the extracellular space. The anchor portion may be at the N-terminal end of the polypeptide, the C-terminal end of the polypeptide, or within the polypeptide construct. In certain embodiments, the anchor portion is near the C-terminal end of the polypeptide, and the N-terminal end of the polypeptide is secreted through a membrane.

In certain embodiments, the portion of the polypeptide to be tested for binding, the potential binding moiety, is exposed to the area where binding would typically occur, such as the periplasmic space or the extracellular space. Examples of anchor portions suitable for attaching the polypeptide to the outer membrane include, but are not limited to, phoA, phoE, Ipp, OmpA, OmpC, OmpF, lamb, pillin, and flagellin. Examples of anchor portions suitable for attaching the polypeptide to the inner membrane include, but are not limited to, hydrophobic cores, immunoglobulin anchor portions, secretory leader portions lacking a site for a leader peptidase, and other inner membrane-bound proteins.

In certain embodiments, the polypeptide includes a linker portion between the potential binding moiety and the anchor portion. In certain embodiments, the linker portion comprises a series of amino acids that separate different portions of the polypeptide. In certain embodiments, the linker portion permits different portions of the polypeptide to interact with other molecules without being constrained by other portions of the polypeptide. In certain embodiments, the linker portion allows each portion of the polypeptide to fold independently. In certain embodiments, the linker portion may be comprised of a series of glycines and/or serines. A non-limiting example of a linker portion is a hinge region of an immunoglobulin.

In certain embodiments, the polypeptide has no anchor portion, and the polypeptide is secreted into the periplasmic space as a free protein. In certain embodiments, the polypeptide is exposed to the periplasmic space and the polypeptide comprises a secretory leader portion that lacks a site for cleavage by a leader peptidase.

In certain embodiments, the sequence encoding the polypeptide includes a translational stop codon that terminates protein synthesis.

Certain Exemplary Vector Construction

In certain embodiments, replication of vector is regulated by controllable elements on the vector. In certain embodiments, vectors possess an origin of replication that can be controlled externally, e.g., through the addition of certain agents to the cell media or through changes in the physical environment of the cell such as light or temperature induction. In certain embodiments, the origin of replication permits vectors to be replicated.

In certain embodiments, vectors may be unable to replicate or phage may be unable to assemble in the absence of a helper phage. In certain embodiments, a vector encodes a protein used for viral assembly that is controlled by a promoter capable of being controlled externally. In certain embodiments, a vector encodes a protein or RNA used for vector replication or transmission of the vector to another host cell.

As a non-limiting example, a phage vector may encode a coat protein that is only expressed when a particular chemical is present. When the host cell with the vector is in a selection phase, the chemical is absent. After selection, when the vector is amplified, the chemical is added to the host cell, the coat protein is expressed, and new phage is assembled.

In certain embodiments, transcription of the nucleotide sequence encoding the potential binding moiety is regulated through one or more known transcriptional regulators. In certain embodiments, the vector sequence comprises an operator sequence and regulation is accomplished by binding of a repressor that turns transcription off. In certain embodiments, the vector sequence comprises an initiator sequence and regulation is accomplished by binding of an activator that turns transcription on.

In certain embodiments, the repressor is produced by the host cell. The repressor may be encoded on the host cell chromosome or it may be encoded on an extrachromosomal element. In certain embodiments, the repressor is encoded by the vector. Exemplary operator sequences for repressor binding include, but are not limited to, lacO from the lac operon, Plo and Pro from the lambda promoter, and AraO from the araBAD promoter.

In certain embodiments, the activator is produced by the host cell. The activator may be encoded by the host cell chromosome or an extrachromosomal element. In certain embodiments, the activator is encoded by the vector. In certain embodiments, the activator is added to the host cells. A non-limiting exemplary initiator sequence is aral from the araBAD operon.

In certain embodiments, the vector comprises a promoter. In certain embodiments, the promoter comprises an RNA polymerase binding site. In certain embodiments, the RNA polymerase binding site comprises a Pribnow box or “−10” region. In certain embodiments, the RNA polymerase binding site comprises a “−23” region. In certain embodiments, the RNA polymerase binding site comprises spacing between the “−10” region and the “−23” region. In certain embodiments, the promoter lacks substantial secondary structure in order to be efficiently transcribed. Non-limiting exemplary promoters may found in the araBAD operon, lac operon, Trp operon, Tac operon, Trc operon T7 operon, pL operon and pR operon.

In certain embodiments, the vector comprises a transcription initiation site. In certain embodiments, the initiation site lacks substantial secondary structure. In certain embodiments, the mRNA transcribed begins with an “A.”

In certain embodiments, the vector comprises a ribosomal binding site (RBS). In certain embodiments, the RBS comprises a Shine-Delgarno sequence. In certain embodiments, the RBS comprises sequences that are complementary to an rRNA within a ribosome, such as, for example, the 23S rRNA. In certain embodiments, the Shine-Delgarno sequence is GGAG. In certain embodiments, the RBS is 3 to 5 bases upstream of the start codon.

In certain embodiments, the portion of the vector that encodes the potential binding moiety comprises a start codon at the 5′ end. In certain embodiments, the start codon encodes a methionine residue. In certain embodiments, the start codon is ATG or GTG. In certain embodiments, the portion of the vector encoding the potential binding moiety comprises a termination codon at the 3′ end. In certain embodiments, the termination codon is a codon that does not encode an amino acid.

In certain embodiments, the vector comprises a transcription termination sequence that substantially terminates transcription. In certain embodiments, transcription termination sequences possess a secondary structure. A non-limiting example of a transcription termination sequence is the BT terminator from Baculovirus toxin.

In certain embodiments, a multiple cloning site is between the nucleic acid encoding the secretory leader and the nucleic acid encoding the linker region. In certain embodiments, the multiple cloning site comprises several restriction enzyme sites. In certain embodiments, the multiple cloning site allows nucleic acids encoding potential binding moieties to be inserted and removed from the vector.

In certain embodiments, the nucleic acid encoding the polypeptide comprises a suppressor stop codon. A suppressor stop codon is suppressed when a vector sequence comprising the suppressor stop codon is in a host that possesses a suppressor tRNA that translates an amino acid instead of terminating translation. When the vector sequence is in a host that lacks the suppressor tRNA, the suppressor stop codon acts like a normal stop codon and terminates translation.

In certain embodiments, a suppressor stop codon is between the nucleic acids encoding the potential binding moiety and the anchor portion. In certain embodiments, when the suppressor stop codon is suppressed, the polypeptide is translated so that the potential binding moiety and the anchor portion are both translated. In certain embodiments, when the suppressor stop codon is not suppressed the polypeptide is translated such that the potential binding moiety, but not the anchor portion, is translated. In certain embodiments, the suppressor stop codon directly follows the nucleic acid encoding the C-terminal end of the potential binding moiety. In certain embodiments, the suppressor stop codon is within the nucleic acid encoding the linker region. In certain embodiments, the suppressor stop codon is in the nucleic acid encoding the N-terminal end of the anchor portion.

In certain embodiments, the suppressor stop codon is suppressed during the selection phase of a method to identify potential binding moieties, and is not suppressed during a subsequent amplification phase.

Certain Exemplary Vectors

In certain embodiments, vectors comprise a nucleic acid sequence encoding a potential binding moiety. Exemplary vectors include, but are not limited to, plasmids, cosmids, episomes, other extrachromosomal elements, bacteriophage genomes, viral genomes, phage genomes, genomes of viruses that infect prokaryotic cells, modified extrachromosomal nucleic acid which may be packaged into a phage for subsequent infection, genomes of viruses that infect eukaryotic cells, genomes of viruses that infect insect cells, genomes of baculoviruses, genomes of viruses that infect plant cells, genomes of viruses that infect yeast, and genomes of viruses that infect archaebacteria. In certain embodiments, a vector comprising a nucleic acid encoding a polypeptide, carbohydrate, or other molecule that is presented on the surface or in an accessible compartment of a cell.

In certain embodiments, vectors are included in viruses or phage that are able to infect cells. In certain embodiments, vectors are capable of virus production and encode viral proteins. In certain embodiments, vectors are capable of phage production and encode phage protein. In certain embodiments, during expression of the polypeptide, the cell is not lysed by the virus. In certain embodiments, the virus or phage produced by the vector is capable of being released from the infected cell.

Many viruses or phages have two phases during their replication cycle. In certain instances, the first phase is the lytic phase of the cycle. During the lytic phase of the cycle, a phage inside a cell will typically produce several copies of the phage genome, produce several copies of the phage proteins, and assemble new phage. In certain instances, the presence of several copies of the phage inside a cell will cause the cell to lyse or break open, killing the cell and leaving several copies of the phage.

In certain instances, the second phase is the non-lytic or lysogenic phase of the cycle. In certain instances, during the non-lytic phase of a two phase replication, the phage does not produce additional copies of the phage genome, but may produce proteins encoded in the phage genome. Certain viruses and phage do not have such a two phase replication cycle. Certain such viruses and phages may always be non-lytic. In certain such instances, the phage may produce additional copies of the phage genome, produce additional copies of the phage proteins, and assemble new phage. The presence of such additional phage, however, still does not result in cell lysis.

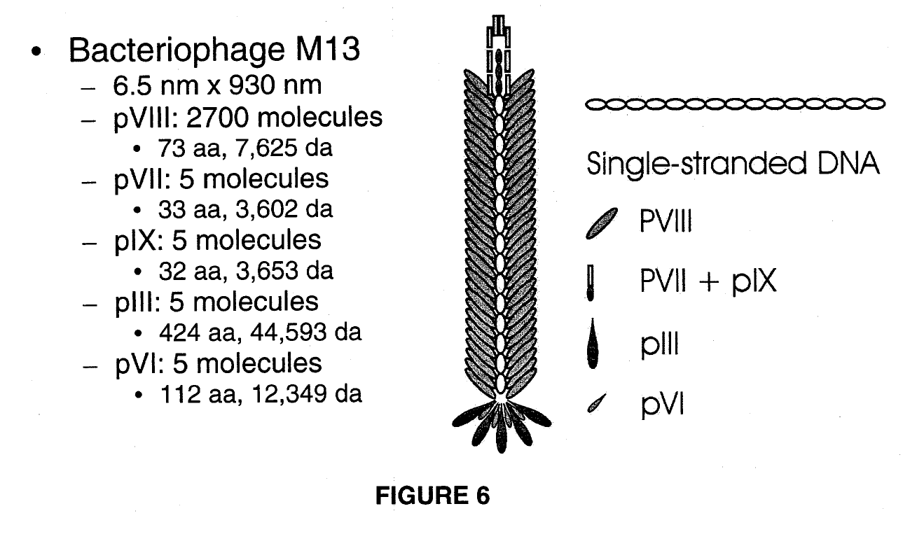

In certain embodiments, the vector may be a non-lytic phage vector of E. coli such as fd or M13. FIG. 6 illustrates an exemplary bacteriophage M13 according to certain embodiments. In certain embodiments, the vector is a two-phase (lytic/non-lytic) phage vector, such as lambda, that is non-lytic during at least a portion of the selection phase of a process for identifying potential binding moieties.

In certain embodiments, a single type of vector is used throughout the method. In certain embodiments of identifying a potential binding moiety, one vector type is used for a selection phase and another vector type is used for a subsequent amplification phase.

In certain embodiments, the vector is not able to produce or assemble phage by itself. As a non-limiting example, one may use a vector of a two phase (lytic/non-lytic) phage, such as lambda, that has been disabled by removing from the vector the nucleic acid sequence that encodes a coat protein. In certain embodiments, since the vector does not produce the coat protein, the vector does not cause the cell to lyse during a selection phase of a process for identifying a binding moiety. In certain embodiments, during the subsequent amplification phase, one adds the coat protein or nucleic acid encoding the coat protein in order to assemble phage and amplify the vector. In certain embodiments, the coat protein may be encoded by another vector, such as another phage vector or phagemid. In certain embodiments, a second helper phage or phagemid is employed to allow the vector to replicate and form phage.

In certain embodiments, vectors include, but are not limited to, plasmids, episomes, extrachromosomal elements, and cosmids. In certain embodiments, vectors are able to replicate and be transmitted to a second host cell that lacks the vector. In certain embodiments, vectors encode proteins required for directing transfer of a vector into a second host cell.

In certain embodiments, vectors replicate and produce multiple copies of the nucleic acid encoding the potential binding moiety. In certain embodiments, the vectors are capable of being transferred to a second host cell by conjugation, transduction, or transformation. Exemplary vectors include, but are not limited to, pBR322, pUC plasmids, prokaryotic plasmids, yeast plasmids, plant plasmids, F factor episomes, and F′ factor episomes.

Certain Exemplary Host Cells

In certain embodiments, host cells include a vector. In certain embodiments, host cells have the vector placed directly into the host cell. In certain embodiments, the host cells produce a polypeptide comprising a potential binding moiety encoded by the vector. In certain embodiments, a polypeptide encoded by the vector is presented on the surface of a cell or in an accessible compartment of a cell. In certain embodiments, the host cells are bacteria and the polypeptide is included in the periplasmic space. In certain embodiments, host cells are used to produce the potential binding moiety on a large scale. In certain embodiments, the large scale production of the potential binding moiety is used to identify the potential binding moiety.

In certain embodiments, host cells are bacteria. In certain embodiments, the bacteria are gram-negative or gram-positive. In certain embodiments, the host cells are E. coli. In certain embodiments, the host cells are yeast, including, but not limited to Saccharomyces cerevisiae. In certain embodiments, the host cells are animal cells. In certain embodiments, the host cells are insect cells. In certain embodiments, the host cells are mammalian cell lines. In certain embodiments, the host cells are plant cells. In certain embodiments, the host cells are human cell lines. Exemplary human cell lines include, but not limited to, NIH 3T3 cells, Jurkatt cells, and MCF-7 cells.

Certain Exemplary Embodiments of the Invention

Certain Exemplary Methods

In certain embodiments, a library of nucleic acid sequences encoding potential binding moieties that may bind to a target is used. Exemplary targets include, but are not limited to, protein ligands, receptors, enzymes, small molecules, carbohydrates, fragments of receptor molecules, pharmaceuticals, and other biologically significant molecules.

In certain embodiments, different members of a library of nucleic acid sequences encoding potential binding moieties are included in separate screening vectors. In certain embodiments, one may employ a vector diagramed in FIG. 3. FIG. 3 illustrates a vector comprising an operator, a promoter, transcription initiation region, and a start codon controlling the transcription of mRNA encoding the polypeptide. The vector shown in FIG. 2 encodes a polypeptide comprising, in the direction N-terminal to C-terminal, a secretory leader, a secretory leader peptidase cleavage site, a potential binding moiety, a scaffold, a linker region, and an anchor portion. In certain embodiments, when the vector shown in FIG. 3 is in a host cell in the presence of an activator protein, ribosomes bind to the promoter region and mRNA transcripts are transcribed. In certain embodiments, when in a host cell, the vector results in potential binding moieties being presented on the surface of the host cell that contains the vector. In certain embodiments, one may employ a nucleic acid encoding a polypeptide that further comprises a carrier protein (or which comprises a carrier protein in lieu of a secretory leader). In certain embodiments, the screening vector is capable of normal phage replication when it is in a suitable host bacterial cell.

In certain embodiments, vectors encoding the polypeptide comprising potential binding moieties are assembled into phage. The assembled phage may then be used to infect a suitable host bacterial cell.

In certain embodiments, vectors encoding the polypeptides comprising potential binding moieties are directly transfected into competent host bacterial cells without the use of phage using standard molecular biological techniques. In certain such embodiments, the vectors comprise nucleic acid for at least some elements necessary for phage production.

In certain embodiments, during the selection process, one uses media conditions that will not result in substantial cell lysis.

In certain embodiments, cells containing the vectors are then induced to express polypeptides encoded by the vectors. In certain embodiments, one induces expression by adding a chemical to the host cell media that induces a promoter on the vector. In certain embodiments, one employs a chemical, such as galactose, that induces the host bacterial cell to produce an activator protein that will in turn induce polypeptide expression. In certain embodiments, this is accomplished by binding of the activator protein to a transcription initiator region on the vector.

In certain embodiments, the polypeptides produced from the vectors are presented on the surface of the host bacterial cells, and remain attached to the outer membrane. In certain embodiments, one employs targets that include a label, such as a fluorescent molecule, to select host cells that have expressed potential binding moieties that bind to the target. In certain such embodiments, the host bacterial cells expressing the polypeptides comprising potential binding moieties are exposed to targets. If an expressed polypeptide on the surface of a host bacterial cell binds to a labeled target, then the host bacterial cell carries the label.

In certain embodiments, the host bacterial cells are then sorted using a flow separation device, such as a fluorescence associated cell sorter (FACS). In certain embodiments, the FACS separates those cells that are labeled from those cells that are not labeled.

In certain embodiments, uninfected bacterial cells are exposed to the labeled cells, which are presumed to have expressed a polypeptide that comprises a binding moiety that binds to the target. In certain embodiments, the infected host bacterial cells expressing polypeptides are then induced to a lytic phase of the phage cycle. In certain embodiments, the infected host bacterial cells are subjected to media conditions that induce phage production and cell lysis. In certain embodiments, the host bacterial cell possessing a vector then replicates several phage per cell. In certain embodiments, a single host cell may produce 100-1,000 phage. In certain embodiments, these phage then infect uninfected host bacterial cells. In certain embodiments, the degree of amplification of vectors is monitored by measuring the optical density of the host cells in the media.

The newly infected host bacterial cells may then be screened for potential binding to the target as described above. Selected host bacterial cells may then be subjected to another round of amplification as described above.

In certain embodiments, in the second round of amplification, the selected host bacterial cells may be placed with second uninfected host bacterial cells of a different type than the initially-used bacterial cells. For example, in certain embodiments, the second host bacterial cells may produce a peptidase that cleaves the anchor portion of the expressed polypeptide from the potential binding moiety. In such embodiments, a soluble polypeptide would be produced that would be secreted into the media outside the cell.

In certain embodiments, soluble peptides may be desired for convenient isolation of the binding moiety without the cells or phage.

In certain embodiments, a suppressor stop codon may be placed in the linker region of the vector between the nucleic acids encoding the potential binding moiety and the nucleic acids encoding the anchor portion. In certain such embodiments, the first host bacterial cell produces a suppressor tRNA. The suppressor tRNA, when it recognizes a specific termination codon (the suppressor stop codon), translates the codon as an amino acid. Typically, where a suppressor tRNA is not present, the suppressor stop codon would signal a termination of translation. In the first host bacterial cell with a suppressor tRNA, a full length polypeptide comprising the anchor portion is produced. In certain embodiments, the second host bacterial cells lack a suppressor tRNA. Accordingly, in such embodiments in the second host bacterial cells, translation is terminated, which results in production of polypeptides comprising the potential binding moiety, but lacking the anchor portion. Such polypeptides would be secreted into the media as soluble polypeptides.

In certain such embodiments, the infected second host bacterial cells are cultured and the soluble polypeptide is expressed in the second host bacterial cells. In certain embodiments, the soluble polypeptide is separated from the cells and phage. Exemplary separation techniques include, but are not limited to, filtration, dialysis, or an affinity purification step. In certain embodiments, this results in production of large amounts of a polypeptide comprising a binding moiety. In certain embodiments, the amount of time required to accomplish production of such large quantities of polypeptide is several hours, with no overnight growth steps.

In certain embodiments, present methods may provide an advantage over certain types of cell surface display methods. In certain instances, cell surface display methods may result in a bias against potential binding moieties if the potential binding moieties negatively impact cell growth. In certain embodiments of the present invention, one need not grow the cells to the extent typically employed in cell surface display techniques such that there is little or no bias against potential binding moieties that would negatively impact cell growth.

Apparatus

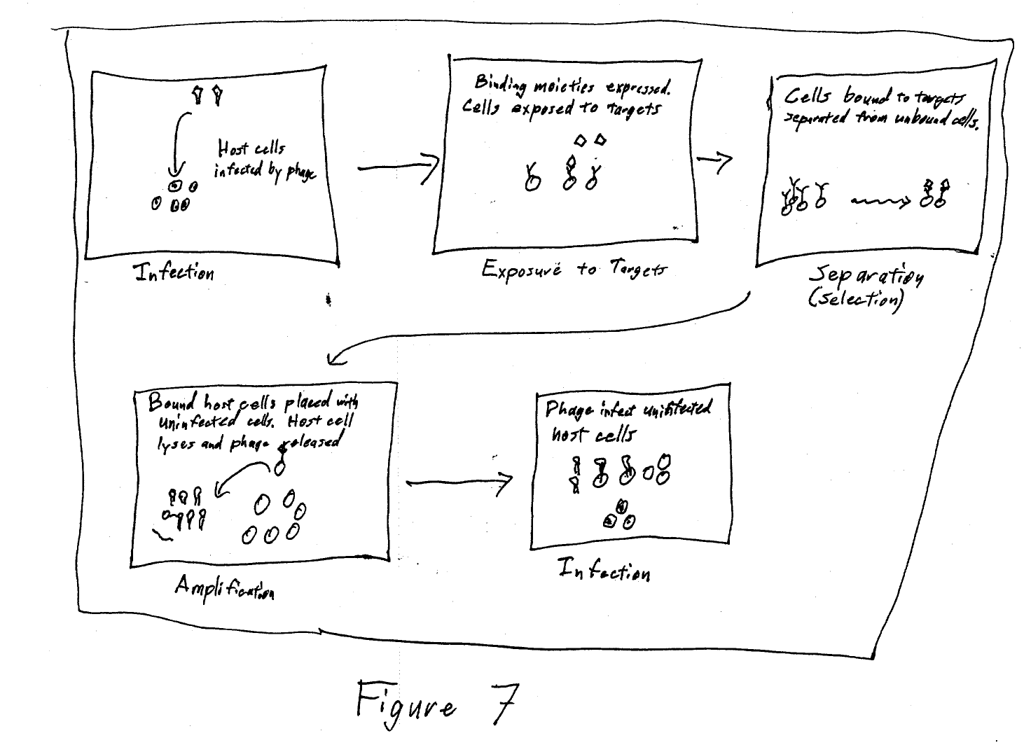

In certain embodiments, an apparatus is provided that can be used to perform selection, separation, and subsequent amplification. In certain embodiments, the apparatus comprises a component that results in first host cells that comprise vectors encoding polypeptides encoding potential binding moieties. In certain embodiments, first host cells comprising vectors encoding polypeptides encoding potential binding moieties express potential binding moieties that are presented on the surface of first host cells. In certain embodiments, the apparatus comprises a component that exposes the first host cells with potential binding moieties on their surface to targets. In certain embodiments, the apparatus comprises a component that separates at least one first host cell that binds to a target from the first host cells that are not bound to targets. In certain embodiments, the apparatus comprises a component that places a separated at least one first host cell into a culture of second host cells that do not comprise nucleic acid encoding potential binding moieties. Exemplary components for separating at least one first host cell with a potential binding moiety bound to a target from first host cells that are not bound to targets include, but are not limited to, traveling wave dielectrophoresis, electrophoresis, diamagnetism, fluorescence-activated cell sorting, dialysis, sedimentation, and centrifugation. In certain embodiments, in the apparatus, phage comprising the vector replicates and infects new host cells. Certain exemplary apparatuses are schematically shown in FIG. 7.

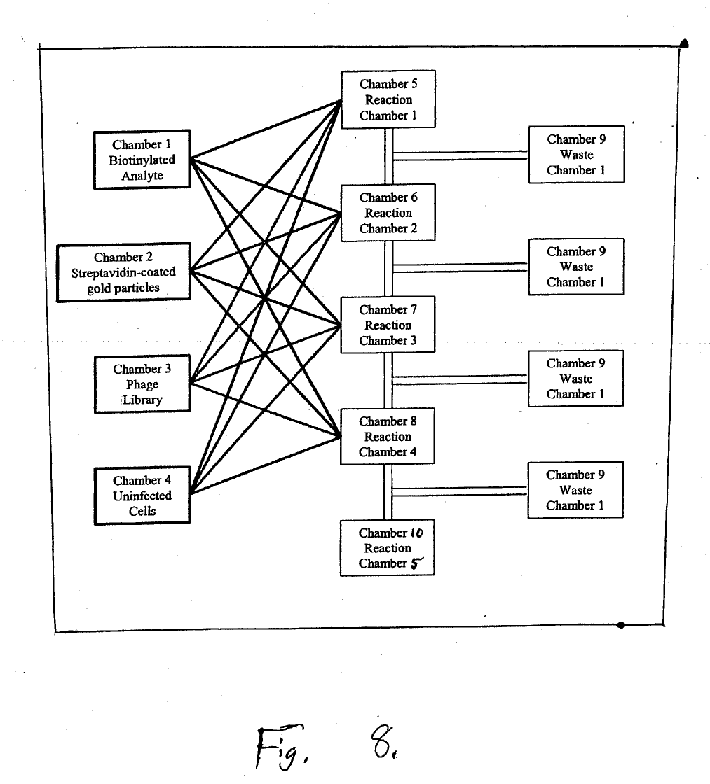

FIG. 8 illustrates how different chambers in an exemplary apparatus may work according to certain embodiments. In FIG. 8, heavy lines represent fluid flow paths. Double lines represent fluid flow paths subjected to electrodes used for carrying out traveling wave dielectrophoresis. In certain embodiments, the electrodes may be in the fluid paths. In certain embodiments, the electrodes may be placed at either end of the fluid paths. The movement of cells from one chamber to another in certain embodiments depicted in FIG. 8 is accomplished by fluid flow or by electrophoresis combined with separation of cells attached to gold nanoparticles by traveling wave dielectrophoresis.

In FIG. 8, uninfected cells (first host cells) from Chamber 4 are moved into Chamber 5 (Reaction Chamber 1). A phage library encoding potential binding moieties is moved from Chamber 3 to Chamber 5, where the phage infect the uninfected cells. In Chamber 5, cells express potential binding moieties. Biotinylated analytes (targets) are moved from Chamber 1 to Chamber 5. Potential binding moieties are exposed to the targets in Chamber 5. Streptavidin-coated gold particles are moved from Chamber 2 to Chamber 5. Cells with binding moieties bound to targets are exposed to the streptavidin-coated gold particles in Chamber 5. Some cells with binding moieties bound to targets will be attached to the gold particles through a biotin-streptavidin bond.

Electrophoresis is used to move the gold particles from Chamber 5 to Chamber 6 (Reaction Chamber 2). The cells without binding moieties bound to targets attached to streptavidin-coated gold particles are moved to Chamber 9 (Waste Chamber 1).

The cells in Chamber 6 are placed in a condition in which the phage in the cells replicate (for example, by changing the medium in which the cells are placed). Uninfected cells (second host cells) from Chamber 4 are moved to Chamber 6, where the newly replicated phage infect the uninfected cells. The replication of phage and phage infection of uninfected cells results in amplification of vectors comprising nucleic acids encoding potential binding moieties. The cells express the potential binding moieties in Chamber 6. The process of selection and separation to the next Chamber (Chamber 7) is repeated, where the amplification of the vectors (comprising phage replication and phage infection of uninfected cells) is also repeated. In certain embodiments, the selection and amplification process may be repeated as many times as desired to select binding moieties that bind the target. See, e.g., the additional Chamber 8 and Chamber 10 (Reaction Chamber 4 and Reaction Chamber 5).

What is claimed is:

1. A method of identifying potential binding moieties that bind to a target comprising:
a) incubating first host cells comprising vectors comprising a library of nucleic acids encoding potential binding moieties to express the potential binding moieties such that the binding moieties are presented on the surface of the first host cells;
b) exposing the first host cells with potential binding moieties on their surface to targets;
c) selecting at least one first host cell with a potential binding moiety on its surface bound to a target;
d) allowing the nucleic acid encoding the potential binding moiety to amplify in a manner that does not require cell division, said amplifying comprising:
exposing second host cells, that do not comprise nucleic acids encoding the potential binding moieties, to a selected at least one first host cell; and
transmitting the nucleic acid encoding the potential binding moiety to at least one second host cell; and
e) identifying the potential binding moiety that binds the target.

2. The method of claim 1, wherein the amplifying comprises allowing the nucleic acid encoding the potential binding moiety to replicate in the at least one first host cell.

3. The method of claim 1, wherein the amplifying comprises allowing the nucleic acid encoding the potential binding moiety to replicate in the at least one second host cell.

4. The method of claim 1, wherein the amplifying comprises allowing the nucleic acid encoding the potential binding moiety to replicate in the at least one first host cell and the at least one second host cell.

5. The method of claim 1, wherein the selecting at least one first host cell with a potential binding moiety on its surface bound to a target comprises separating at least one first host cell with a potential binding moiety on its surface bound to a target from first host cells that do not comprise a potential binding moiety on their surface bound to a target.

6. The method of claim 5, wherein the separating at least one first host cell with a potential binding moiety on its surface bound to a target comprises exposing the at least one first host cell to traveling wave dielectrophoresis.

7. The method of claim 5, wherein the separating at least one first host cell with a potential binding moiety on its surface bound to a target comprises exposing the at least one first host cell to magnetism.

8. The method of claim 5, wherein the separating at least one first host cell with a potential binding moiety on its surface bound to a target comprises exposing the at least one first host cell to flow cytometry.

9. The method of claim 5, wherein the separating at least one first host cell with a potential binding moiety on its surface bound to a target comprises exposing the at least one first host cell to fluorescence-activated cell sorting.

10. The method of claim 1, wherein, after the nucleic acid encoding the potential binding moiety is transmitted to the second host cells, the second host cells are subjected to a subsequent round of a selection comprising:
incubating the second host cells to express potential binding moieties such that the potential binding moieties are presented on the surface of the second host cells;
exposing the second host cells with potential binding moieties on their surface to targets; and
selecting at least one second host cell with a potential binding moiety on its surface bound to a target.

11. The method of claim 1, wherein the second host cells are a different type of cell from the first host cells.

12. The method of claim 11, wherein the second host cells express the potential binding moiety in soluble form.

13. The method of claim 1, further comprising:
exposing the first host cells with potential binding moieties on their surface to blocking molecules after the incubating the first host cells to express potential binding moieties such that the potential binding moieties are presented on the surface of the first host cells, and before the exposing the first host cells with potential binding moieties on their surface to targets;
wherein potential binding moieties bound to blocking molecules are inhibited from binding to targets.

14. The method of claim 1, wherein the allowing the nucleic acid encoding the potential binding moieties to amplify comprises phage amplification.

15. The method of claim 1, wherein the allowing the nucleic acid encoding the potential binding moiety to amplify comprises amplification employing a helper phage.

16. The method of claim 1, wherein the allowing the nucleic acid encoding the potential binding moiety to amplify comprises:
allowing the nucleic acid encoding the potential binding moiety to replicate; and
allowing the nucleic acid encoding the potential binding moiety to assemble into a transmitting agent.

17. The method of claim 16, wherein the allowing the nucleic acid encoding the potential binding moiety to assemble into transmitting agents comprises viral assembly into a phage.

18. The method of claim 17, wherein the transmitting the nucleic acid encoding the potential binding moiety to at least one second host cell comprises infection of the at least one second host cell by the phage.

19. The method of claim 1, further comprising:
exposing the first host cells with potential binding moieties on their surfaces to blocking molecules after the expressing potential binding moieties such that the binding moieties are presented on their surfaces of the first host cells, and before the exposing the first host cells with potential binding moieties on their surfaces to targets;
wherein the potential binding moieties bound to blocking molecules are inhibited from binding to targets.

20. The method of claim 1, wherein the targets are attached to a label.

21. The method of claim 20, wherein the label is biotin.

22. The method of claim 21, further comprising exposing the at least one first host cell with a potential binding moiety on its surface bound to a target to magnetic particles attached to streptavidin.

23. The method of claim 22, wherein the selecting at least one first host cell with a potential binding moiety on its surface bound to a target comprises:
subjecting first host cells to a magnetic field, and
separating the at least one first host cell with a potential binding moiety on its surface bound to a target from other first host cells.

24. The method of claim 20, wherein the selecting at least one first host cell with a potential binding moiety on its surface bound to a target comprises separating the at least one first host cell with a potential binding moiety on its surface bound to a target from other first host cells using a separating label.

25. The method of claim 24, wherein the separating label is selected from at least one of a latex particle, a paramagnetic particle, a particle with specific dielectrophoretic properties, a mobility modifier, and a charged particle.

26. The method of claim 24, wherein the separating label is a first member of an affinity binding set, wherein the first member of the affinity binding set is capable of binding to a second member of the affinity binding set.

27. The method of claim 26, wherein the separating label is selected from biotin, fluorescein, calmodulin binding peptide, rhodamine, and digoxygenin.

28. The method of claim 26, wherein the second member of the affinity binding set is selected from streptavidin, anti-fluorescein antibody, calmodulin, anti-rhodamine antibody, and anti-digoxygenin antibody.

29. The method of claim 28, wherein the second member of the affinity binding set is attached to at least one of a latex particle, a paramagnetic particle, a particle with specific dielectrophoretic properties, a mobility modifier, and a charged particle.

30. The method of claim 1, wherein the selecting at least one first host cell with a potential binding moiety on its surface bound to a target comprises selecting at least two first host cells with potential binding moieties on their surface bound to targets; and
wherein the allowing the nucleic acid encoding the potential binding moiety to amplify in a manner that does not require cell division comprises allowing the nucleic acids encoding the potential binding moieties to amplify with insubstantial cell division.

31. A method of identifying potential binding moieties that bind to a target comprising:
a) incubating first host cells comprising vectors comprising a library of nucleic acids encoding potential binding moieties to express the potential binding moieties such that the potential binding moieties are presented in an accessible compartment of the first host cells;
b) exposing the first host cells with potential binding moieties in their accessible compartments to targets;
c) selecting at least one first host cell with a potential binding moiety bound to a target;
d) allowing the nucleic acid encoding the potential binding moiety to amplify in a manner that does not require cell division, said amplifying comprising:
exposing second host cells, that do not comprise nucleic acids encoding the potential binding moieties, to a selected at least one first host cell; and
transmitting the nucleic acid encoding the potential binding moiety to the second host cells; and
e) identifying the potential binding moiety that binds the target.

32. The method of claim 32, wherein the amplifying comprises allowing the nucleic acid encoding the potential binding moiety to replicate in the at least one first host cell.

33. The method of claim 32, wherein the amplifying comprises allowing the nucleic acid encoding the potential binding moiety to replicate in the at least one second host cell.

34. The method of claim 32, wherein the amplifying comprises allowing the nucleic acid encoding the potential binding moiety to replicate in the at least one first host cell and the at least one second host cell.

35. The method of claim 31, wherein the selecting at least one first host cell with a potential binding moiety in its accessible compartment bound to a target comprises separating at least one first host cell with a potential binding moiety in its accessible compartment bound to a target from first host cells that do not comprise potential binding moieties in their accessible compartments bound to targets.

36. The method of claim 35, wherein the separating at least one first host cell with a potential binding moiety in its accessible compartment bound to a target comprises exposing the at least one first host cell to traveling wave dielectrophoresis.

37. The method of claim 35, wherein the separating at least one first host cell with a potential binding moiety in its accessible compartment bound to a target comprises exposing the at least one first host cell to magnetism.

38. The method of claim 35, wherein the separating at least one first host cell with a potential binding moiety in its accessible compartment bound to a target comprises exposing the at least one first host cell to flow cytometry.

39. The method of claim 35, wherein the separating at least one first host cell with a potential binding moiety in its accessible compartment bound to a target comprises exposing the at least one first host cell to fluorescence-activated cell sorting.

40. The method of claim 31, wherein, after the nucleic acid encoding the potential binding moiety is transmitted to the second host cells, the second host cells are subjected to a subsequent round of a selection comprising:
incubating the second host cells to express potential binding moieties such that the potential binding moieties are presented in an accessible compartment of the second host cells;
exposing the second host cells with potential binding moieties in their accessible compartments to targets;
selecting at least one second host cell with a potential binding moiety in its accessible compartment bound to a target.

41. The method of claim 31, wherein the potential binding moiety presented in the accessible compartment of the first host cells is attached to the inner membrane of the host cells.

42. The method of claim 31, wherein the potential binding moiety presented in the accessible compartment of the first host cells is attached to the outer membrane of the first host cells.

43. The method of claim 31, wherein the potential binding moiety presented in the accessible compartment of the first host cells is not attached to either the inner membrane or the outer membrane of the first host cells.

44. The method of claim 31, wherein the allowing the nucleic acid encoding the potential binding moiety to amplify comprises:
allowing the nucleic acid encoding the potential binding moiety to replicate; and
allowing the nucleic acid encoding the potential binding moiety to assemble into a transmitting agent.

45. The method of claim 44, wherein the allowing the nucleic acid encoding the potential binding moiety to assemble into transmitting agents comprises viral assembly into a phage.

46. The method of claim 45, wherein the transmitting the nucleic acid encoding the potential binding moiety to at least one second host cell comprises infection of the at least one second host cell by the phage.

47. The method of claim 31, wherein the first host cells express the potential binding moiety in soluble form.

48. The method of claim 31, wherein the second host cells are a different type of cell from the first host cells.

49. The method of claim 48, wherein the potential binding moiety presented in the accessible compartment of the second host cells is attached to the inner membrane of the host cells.

50. The method of claim 49, wherein the potential binding moiety presented in the accessible compartment of the second host cells is attached to the outer membrane of the first host cells.

51. The method of claim 48, wherein the potential binding moiety presented in the accessible compartment of the second host cells is not attached to either the inner membrane or the outer membrane of the first host cells.

52. The method of claim 48, wherein the second host cells express the potential binding moiety in soluble form.

53. The method of claim 31, further comprising:
exposing the first host cells with potential binding moieties in their accessible compartments to blocking molecules after the incubating the first host cells to express potential binding moieties such that the potential binding moieties are presented in the accessible compartments of the first host cells, and before the exposing the first host cells with potential binding moieties in their accessible compartments to targets;
wherein the potential binding moieties bound to blocking molecules are inhibited from binding to targets.

54. The method of claim 31, wherein the allowing the nucleic acid encoding the potential binding moiety to amplify comprises phage amplification.

55. The method of claim 31, wherein the targets are attached to a label.

56. The method of claim 55, wherein the label is biotin.

57. The method of claim 56, further comprising exposing the at least one first host cell with a potential binding moiety in an accessible compartment bound to a target to magnetic particles attached to streptavidin.

58. The method of claim 57, wherein the selecting at least one first host cell with a potential binding moiety in an accessible compartment bound to a target comprises:
subjecting first host cells to a magnetic field, and
separating the at least one first host cell with a potential binding moiety in an accessible compartment to a target from other first host cells.

59. The method of claim 55, wherein the selecting at least one first host cell with a potential binding moiety in an accessible compartment bound to a target comprises separating the at least one first host cell with a potential binding moiety in an accessible compartment bound to a target from other first host cells using a separating label.

60. The method of claim 31, wherein the selecting at least one first host cell with a potential binding moiety in its accessible compartment bound to a target comprises selecting at least two first host cells with potential binding moieties in their accessible compartments bound to targets; and
wherein the allowing the nucleic acid encoding the potential binding moiety to amplify in a manner that does not require cell division comprises allowing the nucleic acids encoding the potential binding moieties to amplify with insubstantial cell division.

61. A method of identifying potential binding moieties that bind to a target comprising:
a) incubating first host cells comprising vectors comprising a library of nucleic acids encoding potential binding moieties to express the potential binding moieties such that the binding moieties are presented on the surface of the first host cells;
b) exposing the first host cells with potential binding moieties on their surface to targets;
c) selecting at least one first host cell with a potential binding moiety on its surface bound to a target;
d) allowing the nucleic acid encoding the potential binding moiety to amplify,
said amplifying comprising:
exposing a culture of second host cells, that do not comprise nucleic acids encoding the potential binding moieties, to a selected at least one first host cell; and
transmitting the nucleic acid encoding the potential binding moiety to second host cells; and
e) identifying the potential binding moiety that binds the target.

62. The method of claim 61, wherein the amplifying comprises allowing the nucleic acid encoding the potential binding moiety to replicate in the at least one first host cell.

63. The method of claim 61, wherein the amplifying comprises allowing the nucleic acid encoding the potential binding moiety to replicate in the at least one second host cell.

64. The method of claim 61, wherein the amplifying comprises allowing the nucleic acid encoding the potential binding moiety to replicate in the at least one first host cell and the at least one second host cell.

65. The method of claim 61, wherein the selecting at least one first host cell with a potential binding moiety on its surface bound to a target comprises separating at least one first host cell with a potential binding moiety on its surface bound to a target from first host cells that do not comprise a potential binding moiety on their surface bound to a target.

66. The method of claim 65, wherein the separating at least one first host cell with a potential binding moiety on its surface bound to a target comprises exposing the at least one first host cell to traveling wave dielectrophoresis.

67. The method of claim 65, wherein the separating at least one first host cell with a potential binding moiety on its surface bound to a target comprises exposing the at least one first host cell to fluorescence-activated cell sorting.

68. The method of claim 61, wherein, after the nucleic acid encoding the potential binding moiety is transmitted to the second host cells, the second host cells are subjected to a subsequent round of a selection comprising:
incubating the second host cells to express the potential binding moieties such that the binding moieties are presented on the surface of the second host cells;
exposing the second host cells with potential binding moieties on their surface to targets; and
selecting at least one second host cell with a potential binding moiety on its surface bound to a target.

69. The method of claim 61, wherein the second host cells are a different type of cell from the first host cells.

70. The method of claim 69, wherein the second host cells express the potential binding moiety in soluble form.

71. The method of claim 61, further comprising:
exposing the first host cells with potential binding moieties on their surface to blocking molecules after the incubating the first host cells to express potential binding moieties such that the potential binding moieties are presented on the surface of the first host cells, and before the exposing the first host cells with potential binding moieties on their surface to targets;
wherein potential binding moieties bound to blocking molecules are inhibited from binding to targets.

72. The method of claim 61, wherein the allowing the nucleic acid encoding the potential binding moieties to amplify comprises phage amplification.

73. The method of claim 61, wherein the allowing the nucleic acid encoding the potential binding moiety to amplify comprises:
allowing the nucleic acid encoding the potential binding moiety to replicate; and
allowing the nucleic acid encoding the potential binding moiety to assemble into a transmitting agent.

74. The method of claim 73, wherein the allowing the nucleic acid encoding the potential binding moiety to assemble into transmitting agents comprises viral assembly into a phage.

75. The method of claim 74, wherein the transmitting the nucleic acid encoding the potential binding moiety to at least one second host cell comprises infection of the at least one second host cell by the phage.

76. The method of claim 61, further comprising exposing the at least one first host cell with a potential binding moiety on its surface bound to a target to magnetic particles attached to streptavidin.

77. The method of claim 76, wherein the selecting at least one first host cell with a potential binding moiety on its surface bound to a target comprises:
subjecting first host cells to a magnetic field, and
separating the at least one first host cell with a potential binding moiety on its surface bound to a target from other first host cells.

78. The method of claim 61, wherein the selecting at least one first host cell with a potential binding moiety on its surface bound to a target comprises selecting at least two first host cells with potential binding moieties on their surface bound to targets; and
wherein the allowing the nucleic acid encoding the potential binding moiety to amplify in a manner that does not require cell division comprises allowing the nucleic acids encoding the potential binding moieties to amplify with insubstantial cell division.

79. A method of identifying potential binding moieties that bind to a target comprising:
a) incubating first host cells comprising vectors comprising a library of nucleic acids encoding potential binding moieties to express the potential binding moieties such that the potential binding moieties are presented in accessible compartments of the first host cells;
b) exposing the first host cells with potential binding moieties in their accessible compartments to targets;
c) selecting at least one first host cell with a potential binding moiety bound to a target;
d) allowing the nucleic acid encoding the potential binding moiety to amplify, said amplifying comprising:
exposing a culture of second host cells, that do not comprise nucleic acids encoding the potential binding moieties, to a selected at least one first host cell; and
transmitting the nucleic acid encoding the potential binding moiety to the second host cells; and
e) identifying the potential binding moiety that binds the target.

80. The method of claim 79, wherein the amplifying comprises allowing the nucleic acid encoding the potential binding moiety to replicate in the at least one first host cell.

81. The method of claim 79, wherein the amplifying comprises allowing the nucleic acid encoding the potential binding moiety to replicate in the at least one second host cell.

82. The method of claim 79, wherein the amplifying comprises allowing the nucleic acid encoding the potential binding moiety to replicate in the at least one first host cell and the at least one second host cell.

83. The method of claim 79, wherein the selecting at least one first host cell with a potential binding moiety in its accessible compartment bound to a target comprises separating at least one first host cell with a potential binding moiety in its accessible compartment bound to a target from first host cells that do not comprise potential binding moieties in their accessible compartments bound to targets.

84. The method of claim 83, wherein the separating at least one first host cell with a potential binding moiety in its accessible compartment bound to a target comprises exposing the at least one first host cell to traveling wave dielectrophoresis.

85. The method of claim 83, wherein the separating at least one first host cell with a potential binding moiety in its accessible compartment bound to a target comprises exposing the at least one first host cell to fluorescence-activated cell sorting.

86. The method of claim 79, wherein, after the nucleic acid encoding the potential binding moiety is transmitted to the second host cells are, the second host cells are subjected to a subsequent round of a selection comprising:
incubating the second host cells to express the potential binding moieties such that the potential binding moieties are presented in an accessible compartment of the second host cells;
exposing the second host cells with potential binding moieties in their accessible compartments to targets;
selecting at least one second host cell with a potential binding moiety in its accessible compartment bound to a target.

87. The method of claim 79, wherein the potential binding moiety presented in the accessible compartment of the first host cells is attached to the inner membrane of the host cells.

88. The method of claim 79, wherein the potential binding moiety presented in the accessible compartment of the first host cells is attached to the outer membrane of the first host cells.

89. The method of claim 79, wherein the potential binding moiety presented in the accessible compartment of the first host cells is not attached to either the inner membrane or the outer membrane of the first host cells.

90. The method of claim 79, wherein the allowing the nucleic acid encoding the potential binding moiety to amplify comprises:
allowing the nucleic acid encoding the potential binding moiety to replicate; and
allowing the nucleic acid encoding the potential binding moiety to assemble into a transmitting agent.

91. The method of claim 90, wherein the allowing the nucleic acid encoding the potential binding moiety to assemble into transmitting agents comprises viral assembly into a phage.

92. The method of claim 91, wherein the transmitting the nucleic acid encoding the potential binding moiety to at least one second host cell comprises infection of the at least one second host cell by the phage.

93. The method of claim 79, further comprising:
exposing the first host cells with potential binding moieties in their accessible compartments to blocking molecules after the incubating the first host cells to express potential binding moieties such that the potential binding moieties are presented in the accessible compartments of the first host cells, and before the exposing the first host cells with potential binding moieties in their accessible compartments to targets;
wherein the potential binding moieties bound to blocking molecules are inhibited from binding to targets.
